SEQUENCE LISTING (37 sequences)

SEQ ID NO: 1 - DNA, 20 bp, Artificial Sequence
  SNP(rs11105378)_1st_Fw primer
ggcagctacacaggtgttca

SEQ ID NO: 2 - DNA, 20 bp, Artificial Sequence
  SNP(rs11105378)_1st_Rv primer
cgggaaaacagcagtcattt

SEQ ID NO: 3 - DNA, 20 bp, Artificial Sequence
  SNP(rs11105378)_SS Primer_Fw(C) primer
gctagtctgtttttcatggc

SEQ ID NO: 4 - DNA, 20 bp, Artificial Sequence
  SNP(rs11105378)_SS Primer_Fw(T) primer
gctagtctgtttttcatggt

SEQ ID NO: 5 - DNA, 21 bp, Artificial Sequence
  SNP(rs11105378)_AS Primer_Fw(C) primer
gctagtctgtttttcatgaca

SEQ ID NO: 6 - DNA, 21 bp, Artificial Sequence
  SNP(rs11105378)_AS Primer_Fw(T) primer
gctagtctgtttttcatgata

SEQ ID NO: 7 - DNA, 20 bp, Artificial Sequence
  SNP(rs11105378)_Rv primer
cgggaaaacagcagtcattt

SEQ ID NO: 8 - DNA, 20 bp, Artificial Sequence
  SNP(rs2681472)_1st_Fw primer
tctgaggatgtggcatttga

SEQ ID NO: 9 - DNA, 20 bp, Artificial Sequence
  SNP(rs2681472)_1st_Rv primer
tagccacactggcctctttt

SEQ ID NO: 10 - DNA, 20 bp, Artificial Sequence
  SNP(rs2681472)_SS Primer_Fw?A) primer
agtgggtctgccatgtaaat

SEQ ID NO: 11 - DNA, 20 bp, Artificial Sequence
  SNP(rs2681472)_SS Primer_Fw(G) primer
agtgggtctgccatgtaaac

SEQ ID NO: 12 - DNA, 21 bp, Artificial Sequence
  SNP(rs2681472)_AS Primer_Fw(A) primer
agtgggtctgccatgtaagta

SEQ ID NO: 13 - DNA, 21 bp, Artificial Sequence
  SNP(rs2681472)_AS Primer_Fw(G) primer
agtgggtctgccatgtaagca

SEQ ID NO: 14 - DNA, 20 bp, Artificial Sequence
  SNP(rs2681472)_Rv primer
tagccacactggcctctttt

SEQ ID NO: 15 - DNA, 20 bp, Artificial Sequence
  SNP(rs1401982)_1st_Fw primer
tgtggctaggggagcagata

SEQ ID NO: 16 - DNA, 20 bp, Artificial Sequence
  SNP(rs1401982)_1st_Rv primer
aatgctccaccaacaaggtt

SEQ ID NO: 17 - DNA, 20 bp, Artificial Sequence
  SNP(rs1401982)_SS Primer_Fw(G) primer
cctatgttcttggagttatc

SEQ ID NO: 18 - DNA, 20 bp, Artificial Sequence
  SNP(rs1401982)_SS Primer_Fw(A) primer
cctatgttcttggagttatt

SEQ ID NO: 19 - DNA, 21 bp, Artificial Sequence
  SNP(rs1401982)_AS Primer_Fw(G) primer
cctatgttcttggagttaccc

SEQ ID NO: 20 - DNA, 21 bp, Artificial Sequence
  SNP(rs1401982)_AS Primer_Fw(A) primer
cctatgttcttggagttactc

SEQ ID NO: 21 - DNA, 20 bp, Artificial Sequence
  SNP(rs1401982)_Rv primer
aatgctccaccaacaaggtt

SEQ ID NO: 22 - DNA, 20 bp, Artificial Sequence
  SNP(rs1799998)_1st_Fw primer
tggagggtgtacctgtgtca

SEQ ID NO: 23 - DNA, 20 bp, Artificial Sequence
  SNP(rs1799998)_1st_Rv primer
tccagggctgagaggagtaa

SEQ ID NO: 24 - DNA, 20 bp, Artificial Sequence
  SNP(rs1799998)_SS Primer_Fw(T) primer
tattaaaagaatccaaggct

SEQ ID NO: 25 - DNA, 20 bp, Artificial Sequence
  SNP(rs1799998)_SS Primer_Fw(C) primer
tattaaaagaatccaaggcc

SEQ ID NO: 26 - DNA, 21 bp, Artificial Sequence
  SNP(rs1799998)_AS Primer_Fw(T) primer
tattaaaagaatccaaggttc

SEQ ID NO: 27 - DNA, 21 bp, Artificial Sequence
  SNP(rs1799998)_AS Primer_Fw(C) primer
tattaaaagaatccaaggtcc

SEQ ID NO: 28 - DNA, 20 bp, Artificial Sequence
  SNP(rs1799998)_Rv primer
tccagggctgagaggagtaa

SEQ ID NO: 29 - DNA, 20 bp, Artificial Sequence
  SNP(rs699)_1st_Fw primer
gaactggatgttgctgctga

SEQ ID NO: 30 - DNA, 20 bp, Artificial Sequence
  SNP(rs699)_1st_Rv primer
agagccagcagagaggtttg

SEQ ID NO: 31 - DNA, 20 bp, Artificial Sequence
  SNP(rs699)_SS Primer_Fw(T) primer
aagactggctgctccctgat

SEQ ID NO: 32 - DNA, 20 bp, Artificial Sequence
  SNP(rs699)_SS Primer_Fw(M) primer
aagactggctgctccctgac

SEQ ID NO: 33 - DNA, 21 bp, Artificial Sequence
  SNP(rs699)_AS Primer_Fw(T) primer
aagactggctgctccctggtg

SEQ ID NO: 34 - DNA, 21 bp, Artificial Sequence
  SNP(rs699)_AS Primer_Fw(M) primer
aagactggctgctccctggcg

SEQ ID NO: 35 - DNA, 20 bp, Artificial Sequence
  SNP(rs699)_Rv primer
agagccagcagagaggtttg

SEQ ID NO: 36 - DNA, 20 bp, Artificial Sequence
  Primer A
atcctgtcctacctggtaac

SEQ ID NO: 37 - DNA, 20 bp, Artificial Sequence
  Primer B
gtagaaccctgacctaacag

(19) United States
(12) Patent Application Publication
(10) Pub. No.: US 2010/0138943 A1
(43) Pub. Date: Jun. 3, 2010

(54) IDENTIFICATION OF GROUP OF HYPERTENSION-SUSCEPTIBILITY GENES

(75) Inventors: Tetsuro Miki, Toon-shi (JP); Yasuharu Tabara, Matsuyama-shi (JP); Katsuhiko Kohara, Matsuyama-shi (JP); Jun Nakura, Toon-shi (JP); Satoshi Umemura, Yokohama-shi (JP); Nobuhito Hirawa, Yokohama-shi (JP); Hirotsugu Ueshima, Kyoto (JP); Yoshikuni Kita, Kusatsu-shi (JP); Yasuyuki Nakamura, Kyoto (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignees: NAT'L UNIVERSITY CORPORATION EHIME UNIVERSITY, Matsuyama-shi (JP); SHIGA UNIVERSITY OF MEDICAL SCIENCE, Otsu-shi (JP); Public University Corporation Yokohama City University, Yokohama-shi (JP)

(21) Appl. No.: 12/621,235

(22) Filed: Nov. 18, 2009

Related U.S. Application Data
(63) Continuation-in-part of application No. 12/600,223.

(30) Foreign Application Priority Data
Feb. 21, 2008 (JP) 2008-040208

(51) Int. Cl. A01K 67/00; C07H 21/04; C12Q 1/68; C40B 40/08; C12N 15/63

(52) U.S. Cl. 800/9; 536/23.1; 536/24.33; 536/24.3; 435/6; 506/17; 435/320.1; 800/13

(57) ABSTRACT

A genetic marker including a SNP which can be used for assessing the risk of developing hypertension, a polynucleotide for assessing the risk of developing hypertension which can be used as a primer or probe for detecting the genetic marker, a method for assessing the risk of developing hypertension using the SNP, a microarray for assessing the risk of developing hypertension which is used for genotyping of the SNP, a kit used in the method for assessing the risk of developing hypertension, and the like.

This application is a Continuation In-Part application of U.S. patent application Ser. No. 12/600,223, filed Nov. 13, 2009, which claims priority on Japanese Patent Application No. 2008-040208, filed Feb. 21, 2008, and PCT Application No. PCT/JP2009/053012, filed Feb. 20, 2009, which claims priority on Japanese Patent Application No. 2008-040208, the contents of which are incorporated herein by reference.

### BACKGROUND OF THE INVENTION

1. Field of the Invention

The present invention relates to a genetic marker including a SNP which can be used for assessing the risk of developing hypertension, a polynucleotide for assessing the risk of developing hypertension which can be used as a primer or probe for detecting the genetic marker, a method for assessing the risk of developing hypertension using the SNP, a microarray for assessing the risk of developing hypertension which is used for genotyping of the SNP, a kit used in the method for assessing the risk of developing hypertension, and the like.

2. Description of Related Art

High blood pressure is a major factor for the development of, for example, coronary artery disease, cerebral apoplexy (stroke), chronic renal disease, and the like. Accordingly, the prevention of high blood pressure is important in view of social and public health. High blood pressure (hypertension) is a multifactorial disorder, meaning the development thereof may be induced by a number of factors, and these factors inducing high blood pressure are collectively known as risk factors. Risk factors for high blood pressure can be broadly classified into environmental factors and genetic factors, and it is thought that the interactions among these factors play an important role.

Among the risk factors, examples of the environmental factors include aging, obesity, stress, and excessive intake of salt. On the other hand, as the genetic factors, a variety of hypertension-susceptibility genes are known. Here, the term “hypertension-susceptibility gene” refers to a gene which increases the risk of developing hypertension. In other words, people who have (a) hypertension-susceptibility gene(s) are more likely to develop hypertension than those who do not have (a) hypertension-susceptibility gene(s). With respect to multifactorial disorders such as hypertension, it is thought that many of the susceptibility genes are alleles of polymorphisms such as single nucleotide polymorphisms (SNP).

As the hypertension-susceptibility genes containing genetic polymorphisms, various genes have been reported to date including those encoding angiotensinogen, α-adducin, β2-adrenoceptor, glycoprotein Ia (GPIa), chemokine receptor 2 (CCR2), apolipoprotein C (ApoC-III), G-protein β3 subunit (GPβ3), tumor necrosis factor α (TNFα), insulin receptor substrate 1 (IRS-1), glycoprotein Ib α (GPIbα), C-type natriuretic hormone (CNP), heme oxygenase 1 (HMOX-1) and SCNN1A (for example, refer to Patent Documents 1 to 4). Moreover, in recent years, the CYP17 gene (rs6162) polymorphism, the EXOSC3 gene (rs7158) polymorphism, the ACCN1 gene (rs28933) polymorphism, the KCNMB4 gene (rs710652) polymorphism, the KCNIP2 gene (rs755381) polymorphism, the ATP2A3 gene (rs887387) polymorphism, the RAC2 gene (rs929023) polymorphism, the CD3EAP gene (rs967591) polymorphism, the CALCR gene (rs1042138) polymorphism, the ATP10D gene (rs1058793) polymorphism, the GNA14 gene (rs1801258) polymorphism, the PTHR1 gene (rs1869872) polymorphism, the ATP2B1 gene (rs2070759) polymorphism, the HLA-DMB gene (rs2071556) polymorphism, the SLC13A1 gene (rs2140516) polymorphism, the SLC2A11 gene (rs2236620) polymorphism, the GNAI2 gene (rs2236943) polymorphism, the CACNA2D2 gene (rs2236957) polymorphism, the PRKWNK1 gene (rs2255390) polymorphism, the SLC22A7 gene (rs2270860) polymorphism, the KCNN1 gene (rs2278993) polymorphism, the SLC21A6 gene (rs2291075) polymorphism, the CACNA1E gene (rs2293990) polymorphism, the SLC26A8 gene (rs2295852) polymorphism, the ERCC1 gene (rs2298881) polymorphism, the DLGAP2 gene (rs2301963) polymorphism, the COL4A1 gene (rs2305080) polymorphism, the GUCA1C gene (rs2715709) polymorphism, the ATP10C gene (rs3736186) polymorphism, the HCN4 gene (rs3743496) polymorphism, the PTPRT gene (rs3746539) polymorphism, the FGF2 gene (rs3747676) polymorphism, the CHGA gene (rs3759717) polymorphism, the PPP1R1B gene (rs3764352) polymorphism, the ADORA1 gene (rs3766554) polymorphism, the RGS191P1 gene (rs3815715) polymorphism and the RGS20 gene (rs3816772) polymorphism have been reported as hopeful candidates as hypertension-susceptibility gene polymorphisms (for example, refer to Patent Document 5).

An object of the treatment for high blood pressure is to lower the blood pressure in order to prevent the development of coronary artery disease or the like caused by high blood pressure.

The treatment methods can be broadly classified into the reduction of risk factors for hypertension through the improvements of lifestyle habits such as eating habits and the administration of antihypertensive drugs. The risk of developing hypertension for each patient is usually first assessed, followed by determination of the target blood pressure and treatment method based on the assessed results and the patients' actual blood pressure. For example, firstly, based on the patients' (systolic blood pressure)/(diastolic blood pressure), patients are classified as having mild hypertension (from 140 to 159 mmHg)/(from 90 to 99 mmHg), moderate hypertension (from 160 to 179 mmHg)/(from 100 to 109 mmHg) and severe hypertension (≧180 mmHg)/(≧110 mmHg), and then the risk for each patient is assessed with taking risk factors other than blood pressure into account. For instance, patients having mild hypertension with no other risk factors are classified into a low risk group, patients having mild hypertension and several moderate risk factors are classified into an intermediate risk group, and patients having mild hypertension but also having high risk factors such as diabetes are classified into a high risk group. In those cases where patients are in a low risk group or intermediate risk group, they are usually made to first alter their lifestyle habits for a certain period of time, and then put on medication if their blood pressure did not fall satisfactorily. However, the patients in a high risk group are made to alter their lifestyle habits for a certain period of time, while being put on medication at the same time. In other words, even among the patients with comparable blood pressure, treatment methods are not necessarily the same, and an adequate treatment method is appropriately selected depending on the risk factors of each patient. For this reason, it is extremely important to properly evaluate the risk of developing hypertension.

The majority of risk factors do not necessarily pull the triggers for the development of hypertension on their own. Especially in those cases where the hypertension-susceptibility genes classified as the genetic factors are concerned, it has been thought that hypertension develops as a result of the interactions among several hypertension-susceptibility genes that are present. Accordingly, when evaluating the risk of developing hypertension, patients with a large number of hypertension-susceptibility genes are more likely to be classified into a high risk group. Therefore, it is thought that the risk of developing hypertension can be evaluated more properly by examining the presence and absence of as many hypertension-susceptibility genes as possible. However, numerous hypertension-susceptibility genes have already been reported, and it is not preferable to examine all these genes from the viewpoints of both swift evaluation and economic efficiency. In addition, because the correlation between the presence/absence of hypertension-susceptibility genes and the actual development of hypertension differs among various hypertension-susceptibility genes, when a risk assessment is made based on a hypertension-susceptibility gene with which the above-mentioned correlation is relatively low, a highly reliable evaluation cannot be obtained.

[Patent Document 1] Japanese Unexamined Patent Application, First Publication No. 2004-222503

[Patent Document 2] Japanese Unexamined Patent Application, First Publication No. 2004-113094

[Patent Document 3] Japanese Unexamined Patent Application, First Publication No. 2004-33051

[Patent Document 4] Japanese Unexamined Patent Application, First Publication No. 2004-24125

[Patent Document 5] Japanese Unexamined Patent Application, First Publication No. 2007-143504

### PROBLEMS TO BE SOLVED BY THE INVENTION

That is, in order to properly evaluate the risk of developing hypertension, it is preferable to use a useful hypertension-susceptibility gene that is highly correlated with the development of hypertension as a genetic marker. Further, it is more preferable to combine several useful hypertension-susceptibility genes and use them as genetic markers, as long as the number of these genes is within a range so that they can be actually examined clinically. However, regarding the hypertension-susceptibility genes that have been reported to date, although the correlation between their presence/absence and the development of hypertension has been observed, the level of correlation may not be very high, and very few combinations of hypertension-susceptibility genes which may further enhance the reliability of risk assessment have been found.

An object of the present invention is to provide a genetic marker including a SNP whose presence/absence is highly correlated with the development of hypertension and thus can be used for assessing the risk of developing hypertension, a polynucleotide for assessing the risk of developing hypertension which can be used as a primer or probe for detecting the SNP, a method for assessing the risk of developing hypertension using the SNP, a microarray for assessing the risk of developing hypertension which is used for genotyping of the SNP, a kit for assessing the risk of developing hypertension which is used for genotyping of the SNP, and the like.

### SUMMARY OF THE INVENTION

The present inventors have conducted an intensive study in order to solve the above problems as follows and completed the present invention as a result. That is, based on the genomic DNA collected from 8924 subjects, a case-control correlation analysis was conducted examining the differences between SNP frequencies within the case (high blood pressure) group and those within the control (normal blood pressure) group. As a result of the analysis, it was discovered that the SNPs of ATP2B1 and CYP11B2 genes, especially the SNPs of ATP2B1 gene (i.e., rs11105378, rs2681472, rs1401982 and rs11105364) and the SNP of CYP11B2 gene (i.e., rs1799998) were highly useful as genetic markers for hypertension, and that the risk of developing hypertension may be assessed more accurately by combining 2 or more SNPs selected from the group consisting of the above-mentioned SNPs and the SNP of an AGT gene (i.e., rs699) rather than by using individual SNPs alone.

That is, a first aspect of the present invention provides a genetic marker for hypertension, the genetic marker including a sequence homologous to or complementary to the partial or complete sequence of an ATP2B1 gene which contains a single nucleotide polymorphism (SNP) of the ATP2B1 gene, and characterized in that the SNP is at least one SNP selected from the group consisting of a SNP (rs11105378), a SNP (rs2681472), a SNP (rs1401982) and a SNP (rs11105364).

A second aspect of the present invention provides a polynucleotide for assessing the risk of developing hypertension, the polynucleotide including any one of the following base sequences (a) to (f) and characterized in that the polynucleotide can be used as a primer or probe for detecting a SNP (rs11105378):

(a) a base sequence represented by sequence number 5 or a base sequence which is a partial sequence of the base sequence represented by sequence number 5 containing the SNP (rs11105378);

(b) a base sequence complementary to the base sequence (a);

(c) a base sequence composed of the base sequence (a) or (b) in which 1 or more bases other than the SNP (rs11105378) are deleted, substituted, or added, wherein the polynucleotide including the base sequence can be hybridized with the polynucleotide including the base sequence (a) or (b) under stringent conditions;

(d) a base sequence represented by sequence number 6 or a base sequence which is a partial sequence of the base sequence represented by sequence number 6 containing the SNP (rs11105378);

(e) a base sequence complementary to the base sequence (d);

(f) a base sequence composed of the base sequence (d) or (e) in which 1 or more bases other than the SNP (rs11105378) are deleted, substituted, or added, wherein the polynucleotide including the base sequence can be hybridized with the polynucleotide including the base sequence (d) or (e) under stringent conditions.

A third aspect of the present invention provides a polynucleotide for assessing the risk of developing hypertension, the polynucleotide including any one of the following base sequences (a) to (f) and characterized in that the polynucleotide can be used as a primer or probe for detecting a SNP (rs2681472):

(a) a base sequence represented by sequence number 12 or a base sequence which is a partial sequence of the base sequence represented by sequence number 12 containing the SNP (rs2681472);

(b) a base sequence complementary to the base sequence (a);

(c) a base sequence composed of the base sequence (a) or (b) in which 1 or more bases other than the SNP (rs2681472) are deleted, substituted, or added, wherein the polynucleotide including the base sequence can be hybridized with the polynucleotide including the base sequence (a) or (b) under stringent conditions;

(d) a base sequence represented by sequence number 13 or a base sequence which is a partial sequence of the base sequence represented by sequence number 13 containing the SNP (rs2681472);

(e) a base sequence complementary to the base sequence (d);

(f) a base sequence composed of the base sequence (d) or (e) in which 1 or more bases other than the SNP (rs2681472) are deleted, substituted, or added, wherein the polynucleotide including the base sequence can be hybridized with the polynucleotide including the base sequence (d) or (e) under stringent conditions.

A fourth aspect of the present invention provides a polynucleotide for assessing the risk of developing hypertension, the polynucleotide including any one of the following base sequences (a) to (f) and characterized in that the polynucleotide can be used as a primer or probe for detecting a SNP (rs1401982):

(a) a base sequence represented by sequence number 19 or a base sequence which is a partial sequence of the base sequence represented by sequence number 19 containing the SNP (rs1401982);

(b) a base sequence complementary to the base sequence (a);

(c) a base sequence composed of the base sequence (a) or (b) in which 1 or more bases other than the SNP (rs1401982) are deleted, substituted, or added, wherein the polynucleotide including the base sequence can be hybridized with the polynucleotide including the base sequence (a) or (b) under stringent conditions;

(d) a base sequence represented by sequence number 20 or a base sequence which is a partial sequence of the base sequence represented by sequence number 20 containing the SNP (rs1401982);

(e) a base sequence complementary to the base sequence (d);

(f) a base sequence composed of the base sequence (d) or (e) in which 1 or more bases other than the SNP (rs1401982) are deleted, substituted, or added, wherein the polynucleotide including the base sequence can be hybridized with the polynucleotide including the base sequence (d) or (e) under stringent conditions.

A fifth aspect of the present invention provides a genetic marker for hypertension, the genetic marker characterized by including a sequence homologous to or complementary to the partial or complete sequence of a CYP11132 gene containing a SNP (rs1799998) which is a SNP of the CYP11132 gene.

A sixth aspect of the present invention provides a polynucleotide for assessing the risk of developing hypertension, the polynucleotide including any one of the following base sequences (a) to (f) and characterized in that the polynucleotide can be used as a primer or probe for detecting a SNP (rs1799998):

(a) a base sequence represented by sequence number 26 or a base sequence which is a partial sequence of the base sequence represented by sequence number 26 containing the SNP (rs1799998);

(b) a base sequence complementary to the base sequence (a);

(c) a base sequence composed of the base sequence (a) or (b) in which 1 or more bases other than the SNP (rs1799998) are deleted, substituted, or added, wherein the polynucleotide including the base sequence can be hybridized with the polynucleotide including the base sequence (a) or (b) under stringent conditions;

(d) a base sequence represented by sequence number 27 or a base sequence which is a partial sequence of the base sequence represented by sequence number 27 containing the SNP (rs1799998);

(e) a base sequence complementary to the base sequence (d);

(f) a base sequence composed of the base sequence (d) or (e) in which 1 or more bases other than the SNP (rs1799998) are deleted, substituted, or added, wherein the polynucleotide including the base sequence can be hybridized with the polynucleotide including the base sequence (d) or (e) under stringent conditions.

A seventh aspect of the present invention provides a method for assessing the risk of developing hypertension which is a method for assessing the risk of developing hypertension by using a genetic marker, the method characterized by including:

a step (a) of genotyping at least one SNP selected from the group consisting of a SNP (rs11105378), a SNP (rs2681472), a SNP (rs1401982), a SNP (rs11105364) and a SNP (rs1799998) which are present in the nucleic acid molecules collected from a human individual; and

a step (b) of assessing the risk for the human individual to develop hypertension based on the genotyping result obtained in the step (a).

In the seventh aspect of the present invention, it is preferable that the step (a) be a step of genotyping additionally a SNP (rs699) which is a SNP of an AGT gene.

An eighth aspect of the present invention provides a microarray for assessing the risk of developing hypertension, the microarray characterized by including a solid support, and at least one of the polynucleotides for assessing the risk of developing hypertension according to the second, third, fourth and sixth aspects of the present invention which is fixed to the solid support.

In the eighth aspect of the present invention, it is preferable that the polynucleotide which includes any one of the following base sequences (a) to (f) and which can be used as a primer or probe for detecting a SNP (rs699) is also fixed to the solid support:

(a) a base sequence represented by sequence number 33 or a base sequence which is a partial sequence of the base sequence represented by sequence number 33 containing the SNP (rs699);

(b) a base sequence complementary to the base sequence (a);

(c) a base sequence composed of the base sequence (a) or (b) in which 1 or more nucleotides outside the sequence of SNP (rs699) are deleted, substituted, or added, wherein the polynucleotide including the base sequence can be hybridized with the polynucleotide including the base sequence (a) or (b) under stringent conditions;

(d) a base sequence represented by sequence number 34 or a base sequence which is a partial sequence of the base sequence represented by sequence number 34 containing the SNP (rs699);

(e) a base sequence complementary to the base sequence (d);

(f) a base sequence composed of the base sequence (d) or (e) in which 1 or more nucleotides outside the sequence of SNP (rs699) are deleted, substituted, or added, wherein the polynucleotide including the base sequence can be hybridized with the polynucleotide including the base sequence (d) or (e) under stringent conditions.

A ninth aspect of the present invention provides a SNP genotyping kit for assessing the risk of developing hypertension, the SNP genotyping kit characterized by including at least one selected from the group consisting of the polynucleotide for assessing the risk of developing hypertension according to the second aspect of the present invention, the polynucleotide for assessing the risk of developing hypertension according to the third aspect of the present invention, the polynucleotide for assessing the risk of developing hypertension according to the fourth aspect of the present invention, and the polynucleotide for assessing the risk of developing hypertension according to the sixth aspect of the present invention.

In the ninth aspect of the present invention, it is preferable that the SNP genotyping kit includes at least one selected from the group consisting of the polynucleotide for assessing the risk of developing hypertension according to the second aspect of the present invention, the polynucleotide for assessing the risk of developing hypertension according to the third aspect of the present invention, the polynucleotide for assessing the risk of developing hypertension according to the fourth aspect of the present invention, the polynucleotide for assessing the risk of developing hypertension according to the sixth aspect of the present invention, and the polynucleotide including any one of the following base sequences (a) to (f) and which can be used as a primer or probe for detecting a SNP (rs699):

(a) a base sequence represented by sequence number 33 or a base sequence which is a partial sequence of the base sequence represented by sequence number 33 containing the SNP (rs699);

(b) a base sequence complementary to the base sequence (a);

(c) a base sequence composed of the base sequence (a) or (b) in which 1 or more nucleotides outside the sequence of SNP (rs699) are deleted, substituted, or added, wherein the polynucleotide including the base sequence can be hybridized with the polynucleotide including the base sequence (a) or (b) under stringent conditions;

(d) a base sequence represented by sequence number 34 or a base sequence which is a partial sequence of the base sequence represented by sequence number 34 containing the SNP (rs699);

(e) a base sequence complementary to the base sequence (d);

(f) a base sequence composed of the base sequence (d) or (e) in which 1 or more nucleotides outside the sequence of SNP (rs699) are deleted, substituted, or added, wherein the polynucleotide including the base sequence can be hybridized with the polynucleotide including the base sequence (d) or (e) under stringent conditions.

An tenth aspect of the present invention provides a loxP integrated vector, which is used for constructing a small animal in which is an ATP2B1 gene locally deleted, characterized by including a base sequence in which the entire ATP2B1 gene sequence or a portion thereof is sandwiched between loxP sequences.

A eleventh aspect of the present invention provides a loxP integrated small animal, which is used for constructing a small animal in which an ATP2B1 gene is locally deleted, characterized by being constructed using the loxP integrated vector according to the tenth aspect of the present invention.

A twelfth aspect of the present invention provides a small animal in which an ATP2B1 gene is locally deleted, characterized by being constructed through a crossing of a transgenic small animal selectively expressing Cre Recombinase which has at least one promoter selected from the group consisting of a Tie-2 promoter, a Tie-1 promoter, an Flk-1 promoter, an SM22 promoter, an SM-MHC promoter, a Wt1 promoter, a P0 promoter, a Pax3 promoter, an αMHC promoter, an Nkx2.5 promoter, a Tbx1 promoter, a tetracycline-inducible promoter, and a CMV enhancer-chicken β-actin promoter as a promoter for the expression of Cre Recombinase, and the loxP integrated small animal according to the eleventh aspect of the present invention.

A thirteenth aspect of the present invention provides a method for using a small animal in which an ATP2B1 gene is locally deleted, characterized by using the small animal in which the an ATP2B1 gene is locally deleted according to the twelfth aspect of the present invention as a test animal for screening calcium antagonists.

### EFFECT OF THE INVENTION

The genetic marker for hypertension according to the first and/or fifth aspect of the present invention is a genetic marker including a SNP whose presence/absence is highly correlated with the development of hypertension. Accordingly, by using the method for assessing the risk of developing hypertension according to the seventh aspect of the present invention which uses a genetic marker for hypertension according to the present invention, assessed results with higher reliability can be obtained.

In addition, by using any one of the polynucleotides for assessing the risk of developing hypertension according to the second, third, fourth and sixth aspects of the present invention, the microarray for assessing the risk of developing hypertension according to the eighth aspect of the present invention in which the polynucleotides for assessing the risk of developing hypertension are fixed to the solid support, the SNP genotyping kits for assessing the risk of developing hypertension according to the ninth aspect of the present invention including the polynucleotide for assessing the risk of developing hypertension, the SNP which is a genetic marker for hypertension according to the present invention can be detected accurately and easily, and the risk of developing hypertension can be assessed more efficiently with high accuracy.

### BRIEF DESCRIPTION OF THE DRAWINGS

FIG. 1 shows the scheme for generating ATP2B1 foxed mice.

FIG. 2 shows the result of real-time quantitative RT-PCR for determining levels of ATP2B1 mRNA expression of aortas from foxed ATP2B1 and VSMC ATP2B1 KO mice.

FIG. 3 shows the result of measuring blood pressure of conscious homozygous floxed ATP2B1_{loxP/loxP} and VSMC ATP2B1 KO mice.

### DETAILED DESCRIPTION OF THE INVENTION

In the present invention, the term “hypertension (high blood pressure)” refers to a state where the systemic arterial blood pressure transiently or persistently reaches a level so high that disorders such as a cardiovascular system disorder may be induced. There are no particular limitations on the specific definition for the term “hypertension (high blood pressure)”. For example, the term may conform to the Guidelines for the Treatment of Hypertension 2004 (JSH 2004) set out by the Japanese Society of Hypertension, and refers to a state where (systolic blood pressure)/(diastolic blood pressure) is equal to or higher than 140 mmHg/90 mmHg. Among the subjects without taking any antihypertensive agent, in addition to those subjects having the systolic blood pressure equal to or higher than 140 mmHg and the diastolic blood pressure equal to or higher than 90 mmHg, those subjects having the systolic blood pressure less than 140 mmHg but with the diastolic blood pressure equal to or higher than 90 mmHg, and those subjects having the diastolic blood pressure less than 90 mmHg but with the systolic blood pressure equal to or higher than 140 mmHg may also be classified as having a high blood pressure. In addition, hypertension is broadly classified into essential hypertension and secondary hypertension, and 90% or more of patients of hypertension are classified as having essential hypertension. It is preferable that the present invention be used for the patients with essential hypertension.

In the present invention, the term “genetic marker for hypertension” refers to a gene which becomes a marker of the genetic factor for hypertension. The genetic marker for hypertension according to the present invention contains a SNP, and includes a hypertension-susceptibility gene as the allele thereof.

In the present invention, the term “risk of developing hypertension” refers to the liability to hypertension (i.e., possibility of developing hypertension). That is, in the present invention, when a certain individual is classified into a high risk group, this means that it is expected that the individual has a high risk of developing hypertension. On the other hand, when a certain individual is classified into a low risk group, this means that it is expected that the individual has a low risk of developing hypertension.

In the present invention, the SNPs are preferably those SNPs that are registered at public databases and that can be specified from their reference numbers. Examples of such SNPs include the SNPs specified by the rs numbers which are the reference numbers for the SNP database (dbSNP BUILD124) at the National Center for Biotechnology Information (NCBI), and the SNPs specified by the IMS-JST numbers which are the reference numbers for the JSNP (registered trademark) database (http://snp.ims.u-tokyo.ae.jp/index_ja.html), which is a database for the SNPs found among Japanese people and is maintained by the Institute of Medical Science in the University of Tokyo.

The genetic marker for hypertension according to the first aspect of the present invention contains a SNP, and includes an ATP2B1 gene (encoding ATPase, Ca²⁺ transporting, plasma membrane 1) as the allele thereof. More specifically, the genetic marker for hypertension according to the first aspect of the present invention is characterized by including a partial or complete sequence of the ATP2B1 gene which contains, as a SNP of the ATP2B1 gene, at least one SNP selected from the group consisting of a SNP (rs11105378), a SNP (rs2681472), a SNP (rs1401982), and a SNP (rs11105364); and a homologous or complementary sequence thereto. The SNP of the ATP2B1 gene included in the genetic marker may be a single SNP or two or more different SNPs. For example, the SNP may be the SNP (rs11105378) alone, the SNP (rs2681472) alone, the SNP (rs1401982) alone, or the SNP (rs11105364) alone. In addition, the SNP may be a combination of the SNP (rs11105378) and the SNP (rs2681472), a combination of the SNP (rs11105378) and the SNP (rs1401982), a combination of the SNP (rs2681472) and the SNP (rs1401982), and a combination of the SNP (rs11105378), the SNP (rs2681472), the SNP (rs1401982) and the SNP (rs11105364).

ATP2B1 is an enzyme whose expression level is significantly increased among those in the normal blood pressure group and the high blood pressure group, and the ATP2B1 gene is a known hypertension-susceptibility gene. With respect to the human ATP2B1 gene (NCBI Accession Number: NC_{—}000012), for example, although it has been reported that the correlation between a SNP (rs2070759) which is present in the promoter region and that the risk of developing hypertension show significant differences among the respective polymorphisms (for example, refer to Patent Document 5), but the results have not been significant enough to be used for diagnosing the risks. On the other hand, it has been reported that, with respect to 44 patients of essential hypertension (case group) and 40 normotensive subjects having normal blood pressure (control group), all 22 exons of the ATP2B1 gene were subjected to Single Strand Conformation Polymorphism (SSCP) analysis and Heteroduplex (HTX) analysis, and as a result, no significant differences were observed between the case group and the control group even though the sensitivity was 100% (for example, refer to G. R. Monteith et al., Biochemical and Biophysical Research Communications, Vol. 230, No. 2, 1997, pp. 344-346). In other words, although the ATP2B1 gene was a known hypertension-susceptibility gene, the expression level of ATP2B1 gene has been thought to be important for the development of hypertension. The differences in the risk of developing hypertension due to the genetic polymorphisms such as the SNPs other than the promoter region or the like are found only for the first time by the present inventors.

Here, the SNP (rs11105378) is a T/C polymorphism, and as is apparent from the results described later in Example 3, frequency of the C allele is significantly higher than that of the T allele in the high blood pressure group when compared with the normal blood pressure group. Therefore, the SNP (rs11105378) is useful as a genetic marker for hypertension.

In addition, the SNP (rs2681472) is a G/A polymorphism, and as is apparent from the results described later in Example 9, frequency of the A allele is significantly higher than that of the G allele in the high blood pressure group when compared with the normal blood pressure group. Therefore, the SNP (rs2681472) is useful as a genetic marker for hypertension.

Moreover, the SNP (rs1401982) is an A/G polymorphism, and as is apparent from the results described later in Example 15, frequency of the G allele is significantly higher than that of the A allele in the high blood pressure group when compared with the normal blood pressure group. Therefore, the SNP (rs1401982) is useful as a genetic marker for hypertension.

Furthermore, the SNP (rs11105364) is a G/T polymorphism, and as is apparent from the results described later in Example 21, frequency of the T allele is significantly higher than that of the G allele in the high blood pressure group when compared with the normal blood pressure group. Therefore, the SNP (rs11105364) is useful as a genetic marker for hypertension.

The genetic marker for hypertension according to the fifth aspect of the present invention contains a SNP, and includes a CYP11B2 gene (encoding Cytochrome P450, subfamily XIB2) as the allele thereof. More specifically, the genetic marker is characterized by including a sequence homologous to or complementary to the partial or complete sequence of a CYP11B2 gene containing a SNP (rs1799998) which is a SNP of the CYP11B2 gene. The SNP (rs1799998) is a C/T polymorphism, and as is apparent from the results described later in Example 28, frequency of the T allele is significantly higher than that of the C allele in the high blood pressure group when compared with the normal blood pressure group. Therefore, the SNP (rs1799998) is useful as a genetic marker for hypertension. It should be noted that there has been no report showing any particular relationship between the CYP11B2 gene and high blood pressure, and the finding of the CYP11B2 gene being a hypertension-susceptibility gene is made only for the first time by the present inventors.

The method for assessing the risk of developing hypertension according to the seventh aspect of the present invention is a method for assessing the risk of developing hypertension by using a genetic marker for hypertension according to the first or fifth aspect of the present invention (hereinafter, frequently referred to as “genetic marker for hypertension according to the present invention”). More specifically, the seventh aspect of the present invention is characterized by including a step (a) of genotyping at least one SNP selected from the group consisting of the SNP (rs11105378), the SNP (rs2681472), the SNP (rs1401982), the SNP (rs11105364) and the SNP (rs1799998) which are present in the nucleic acid molecules collected from a human individual; and a step (b) of assessing the risk for the human individual to develop hypertension based on the genotyping result obtained in the step (a). Because the tendency to develop hypertension statistically differs significantly among the genotypes with respect to the SNP (rs11105378), the SNP (rs2681472), the SNP (rs1401982), the SNP (rs11105364) and the SNP (rs1799998), it is possible to assess the risk for developing hypertension from the identified genotype of the SNPs.

First, as the step (a), at least one SNP selected from the group consisting of the SNP (rs11105378), the SNP (rs2681472), the SNP (rs1401982), the SNP (rs11105364) and the SNP (rs1799998) which are present in the nucleic acid molecules collected from a human individual is genotyped.

In the present invention, the expression “genotyping of SNP” refers to a procedure in which the base sequence of nucleic acids is analyzed, (an) SNP(s) is/are then detected, and finally (a) polymorphism(s) is/are identified. For example, the procedure detects the SNP (rs11105378) within nucleic acid molecules which is the subject of risk assessment, and identifies which type of polymorphism it is (namely, a TT polymorphism, a TC polymorphism or a CC polymorphism).

There are no particular limitations on the nucleic acids provided for the genotyping of SNPs as long as they are collected from a human individual, and they may be nucleic acids contained in biological samples (specimens) such as blood and body fluids, nucleic acids extracted from these biological samples or the like, or nucleic acids obtained as a result of amplification by using the above-mentioned nucleic acids as templates. In addition, the nucleic acids may be cDNA synthesized from RNA that is contained in biological samples by using a reverse transcriptase.

There are no particular limitations on the method used for SNP genotyping as long as the method is typically used for detecting SNPs. Examples of the methods include the Invader (registered trademark of Third Wave Technologies, Inc.) assay, the Taqman (registered trademark of Applied Biosystems Inc.) assay, MALDI-TOF mass spectrometry, microarray methods, sequence methods, and detection methods using sequence amplification methods such as polymerase chain reactions (PCR). It is particularly desirable that the above method be a process for detecting SNPs by using a primer or a probe which specifically hybridizes with each polymorphism, such as the Taqman assay, PCR methods and microarray methods. For example, in a PCR method, when using a polynucleotide that is completely complementary only with the wild type allele as a wild type primer and using a polynucleotide that is completely complementary only with the mutant allele as a mutant primer, genotypes of SNPs can be identified by carrying out PCR using the nucleic acids containing SNPs as a template, as well as the respective primers, and examining whether or not PCR products are obtained. Similarly, when using a polynucleotide that is completely complementary only with the wild type allele as a wild type probe and using a polynucleotide that is completely complementary only with the mutant allele as a mutant probe, genotypes of SNPs can be identified by using a microarray onto which the nucleic acids containing SNPs are fixed and examining whether or not hybridization occurs when using each of the probes. These methods in which a probe or primer specific to each SNP is used differ from the sequence methods and the like in that SNPs are directly identified. Accordingly, these methods are even more reliable. Additionally, they are also simple and easy since the time required for SNP genotyping is short.

Note that detection of the PCR products which are obtained when PCR is carried out using primers specific to each SNP may be conducted through any method that is typically used when detecting/quantifying PCR products. For example, the PCR products may be detected by electrophoresis, real-time PCR using a fluorescent intercalator such as SYBR Green, or single molecule fluorescence analysis.

There are no particular limitations on the polynucleotide for assessing the risk of developing hypertension which can be used as a primer or probe for detecting a SNP as long as the polynucleotide may hybridize to a partial region of a gene containing the SNP or to a complementary strand thereof. In addition, the sequence length, Tm, and the like with respect to the polynucleotide for assessing the risk of developing hypertension can be determined appropriately by taking genotyping methods, reaction conditions, or the like into consideration. However, the sequence length of the polynucleotide for assessing the risk of developing hypertension is preferably within a range from 10 to 60 bases and more preferably within a range from 15 to 50 bases.

Designing of such polynucleotides for assessing the risk of developing hypertension can be performed by any method known in this field of technology. For example, such polynucleotides can be easily designed by using known genomic sequence data and primer-designing tools used universally. Examples of the primer-designing tools include the Primer3 which can be used on the World Wide Web. In addition, known genomic sequence data can be usually acquired through the international sequence databases such as the National Center for Biotechnology Information (NCBI) and the DNA Data Bank of Japan (DDBJ).

The polynucleotides for assessing the risk of developing hypertension which are designed in such a manner can be synthesized by any method known in this field of technology. For example, such polynucleotides may be custom synthesized by a synthesizing company or may be synthesized independently using a commercially available synthesizer.

As a primer or probe for detecting the SNP (rs11105378) in the ATP2B1 gene, it is particularly desirable to use the polynucleotide for assessing the risk of developing hypertension according to the second aspect of the present invention which includes any one of the following base sequences (a) to (f) (hereinafter, referred to as “polynucleotide for detecting SNP (rs11105378)”):

(a) a base sequence represented by sequence number 5 or a base sequence which is a partial sequence of the base sequence represented by sequence number 5 containing the SNP (rs11105378);

(b) a base sequence complementary to the above-mentioned base sequence (a);

(c) a base sequence composed of the above-mentioned base sequence (a) or (b) in which 1 or more bases other than the SNP (rs11105378) are deleted, substituted, or added, wherein the polynucleotide including the base sequence can be hybridized with the polynucleotide including the above-mentioned base sequence (a) or (b) under stringent conditions;

(d) a base sequence represented by sequence number 6 or a base sequence which is a partial sequence of the base sequence represented by sequence number 6 containing the SNP (rs11105378);

(e) a base sequence complementary to the above-mentioned base sequence (d);

(f) a base sequence composed of the above-mentioned base sequence (d) or (e) in which 1 or more bases other than the SNP (rs11105378) are deleted, substituted, or added, wherein the polynucleotide including the base sequence can be hybridized with the polynucleotide including the above-mentioned base sequence (d) or (e) under stringent conditions.

Note that the polynucleotide for detecting the SNP (rs11105378) having any one of the above-mentioned base sequences (a) to (c) is a polynucleotide which may detect the C allele of the SNP (rs11105378), and the polynucleotide for detecting the SNP (rs11105378) having any one of the above-mentioned base sequences (d) to (f) is a polynucleotide which may detect the T allele of the SNP (rs11105378).

As a primer or probe for detecting the SNP (rs2681472) in the ATP2B1 gene, it is particularly desirable to use the polynucleotide for assessing the risk of developing hypertension according to the third aspect of the present invention which includes any one of the following base sequences (a) to (f) (hereinafter, referred to as “polynucleotide for detecting SNP (rs2681472)”):

(a) a base sequence represented by sequence number 12 or a base sequence which is a partial sequence of the base sequence represented by sequence number 12 containing the SNP (rs2681472);

(b) a base sequence complementary to the above-mentioned base sequence (a);

(c) a base sequence composed of the above-mentioned base sequence (a) or (b) in which 1 or more bases other than the SNP (rs2681472) are deleted, substituted, or added, wherein the polynucleotide including the base sequence can be hybridized with the polynucleotide including the above-mentioned base sequence (a) or (b) under stringent conditions;

(d) a base sequence represented by sequence number 13 or a base sequence which is a partial sequence of the base sequence represented by sequence number 13 containing the SNP (rs2681472);

(e) a base sequence complementary to the above-mentioned base sequence (d);

(f) a base sequence composed of the above-mentioned base sequence (d) or (e) in which 1 or more bases other than the SNP (rs2681472) are deleted, substituted, or added, wherein the polynucleotide including the base sequence can be hybridized with the polynucleotide including the above-mentioned base sequence (d) or (e) under stringent conditions.

Note that the polynucleotide for detecting the SNP (rs2681472) having any one of the above-mentioned base sequences (a) to (c) is a polynucleotide which may detect the A allele of the SNP (rs2681472), and the polynucleotide for detecting the SNP (rs2681472) having any one of the above-mentioned base sequences (d) to (f) is a polynucleotide which may detect the G allele of the SNP (rs2681472).

As a primer or probe for detecting the SNP (rs1401982) in the ATP2B1 gene, it is particularly desirable to use the polynucleotide for assessing the risk of developing hypertension according to the fourth aspect of the present invention which includes any one of the following base sequences (a) to (f) (hereinafter, referred to as “polynucleotide for detecting SNP (rs1401982)”):

(a) a base sequence represented by sequence number 19 or a base sequence which is a partial sequence of the base sequence represented by sequence number 19 containing the SNP (rs1401982);

(b) a base sequence complementary to the above-mentioned base sequence (a);

(c) a base sequence composed of the above-mentioned base sequence (a) or (b) in which 1 or more bases other than the SNP (rs1401982) are deleted, substituted, or added, wherein the polynucleotide including the base sequence can be hybridized with the polynucleotide including the above-mentioned base sequence (a) or (b) under stringent conditions;

(d) a base sequence represented by sequence number 20 or a base sequence which is a partial sequence of the base sequence represented by sequence number 20 containing the SNP (rs1401982);

(e) a base sequence complementary to the above-mentioned base sequence (d);

(f) a base sequence composed of the above-mentioned base sequence (d) or (e) in which 1 or more bases other than the SNP (rs1401982) are deleted, substituted, or added, wherein the polynucleotide including the base sequence can be hybridized with the polynucleotide including the above-mentioned base sequence (d) or (e) under stringent conditions.

Note that the polynucleotide for detecting the SNP (rs1401982) having any one of the above-mentioned base sequences (a) to (c) is a polynucleotide which may detect the G allele of the SNP (rs1401982), and the polynucleotide for detecting the SNP (rs1401982) having any one of the above-mentioned base sequences (d) to (f) is a polynucleotide which may detect the A allele of the SNP (rs1401982).

In addition, as a primer or probe for detecting the SNP (rs1799998) in the CYP11B2 gene, it is particularly desirable to use the polynucleotide for assessing the risk of developing hypertension according to the fifth aspect of the present invention which includes any one of the following base sequences (a) to (f) (hereinafter, referred to as “polynucleotide for detecting SNP (rs1799998)”):

(a) a base sequence represented by sequence number 26 or a base sequence which is a partial sequence of the base sequence represented by sequence number 26 containing the SNP (rs1799998);

(b) a base sequence complementary to the above-mentioned base sequence (a);

(c) a base sequence composed of the above-mentioned base sequence (a) or (b) in which 1 or more bases other than the SNP (rs1799998) are deleted, substituted, or added, wherein the polynucleotide including the base sequence can be hybridized with the polynucleotide including the above-mentioned base sequence (a) or (b) under stringent conditions;

(d) a base sequence represented by sequence number 27 or a base sequence which is a partial sequence of the base sequence represented by sequence number 27 containing the SNP (rs1799998);

(e) a base sequence complementary to the above-mentioned base sequence (d);

(f) a base sequence composed of the above-mentioned base sequence (d) or (e) in which 1 or more bases other than the SNP (rs1799998) are deleted, substituted, or added, wherein the polynucleotide including the base sequence can be hybridized with the polynucleotide including the above-mentioned base sequence (d) or (e) under stringent conditions.

Note that the polynucleotide for detecting the SNP (rs1799998) having any one of the above-mentioned base sequences (a) to (c) is a polynucleotide which may detect the T allele of the SNP (rs1799998), and the polynucleotide for detecting the SNP (rs1799998) having any one of the above-mentioned base sequences (d) to (f) is a polynucleotide which may detect the C allele of the SNP (rs1799998).

It should be noted that in the present invention, the expression “under stringent conditions” means, for example, that the polynucleotide is thermally denatured in a solution containing 5×SSC (150 mM sodium chloride, 15 mM sodium citrate, pH 7.4) and 0.3% SDS (sodium dodecyl sulfate), followed by hybridization at 65° C. for 4 to 16 hours, and the resultant is washed with a solution containing 2×SSC and 0.1% SDS for 5 minutes at room temperature, and then with 2×SSC for 5 minutes, and finally rinsed with 0.05×SSC.

Moreover, in addition to a sequence complementary to or homologous to the gene sequence serving as a detection target, the polynucleotide for assessing the risk of developing hypertension used in the step (a) can include additional base sequences to a degree that SNP genotyping is not inhibited. Examples of the additional base sequences include a restriction site sequence and a sequence provided for labeling nucleic acids. In addition, in order to make the detection or analysis of the results of SNP genotyping simple and easy, a labeling substance can be added to each of the polynucleotides for assessing the risk of developing hypertension to a degree that SNP genotyping is not inhibited. There are no particular limitations on the labeling substance as long as it is a compound typically used for labeling polynucleotides. Examples of the labeling substances include a radioisotope, a fluorescent material, a chemiluminescent material and low molecular compounds such as biotin.

In addition, there are no particular limitations on the quantity of nucleic acids collected from a human individual, polynucleotides for assessing the risk of developing hypertension, and the like when used for genotyping of SNPs, and they can be used in a quantity within a typical range. Moreover, there are no particular limitations on the enzymes such as polymerases, nucleotides, reaction buffers, and the like, and any of these materials typically used when conducting SNP genotyping can be used in a quantity within a typical range.

Subsequently, as the step (b), the risk for the aforementioned human individual to develop hypertension is assessed based on the result of genotyping obtained in the step (a). For example, the risk of developing hypertension may be assessed using the odds ratio indicated in the following Tables 2, 8, 14, 20, and 25. Alternatively, the risk of developing hypertension may be assessed using the odds ratio obtained by conducting a meta-analysis on the genetic markers for hypertension according to the present invention, or may be assessed using the relative risk (risk ratio) obtained by conducting a cohort study on the genetic markers for hypertension according to the present invention, or may even be assessed using other statistical parameters that are treated using conventionally known statistical techniques.

More specifically, when using the genotyping results with respect to the SNP (rs11105378), it is possible to assess that the risk of developing hypertension is highest for the CC genotype, followed by the TC genotype and TT genotype in this order. Alternatively, with respect to the SNP (rs11105378), those with the TT genotype may be assessed as a low risk group whereas those with the TC genotype or CC genotype may be assessed as a high risk group.

When using the genotyping results with respect to the SNP (rs2681472), it is possible to assess that the risk of developing hypertension is highest for the AA genotype, followed by the AG genotype and GO genotype in this order. Alternatively, with respect to the SNP (rs2681472), those with the GG genotype or AG genotype may be assessed as a low risk group whereas those with the AA genotype may be assessed as a high risk group.

When using the genotyping results with respect to the SNP (rs1401982), it is possible to assess that the risk of developing hypertension is highest for the GG genotype, followed by the AG genotype and AA genotype in this order. Alternatively, with respect to the SNP (rs1401982), those with the AA genotype may be assessed as a low risk group whereas those with the AG genotype or GG genotype may be assessed as a high risk group.

When using the genotyping results with respect to the SNP (rs11105364), it is possible to assess that the risk of developing hypertension is highest for the TT genotype, followed by the TG genotype and GG genotype in this order. Alternatively, with respect to the SNP (rs11105364), those with the GG genotype may be assessed as a low risk group whereas those with the TT genotype or TG genotype may be assessed as a high risk group.

On the other hand, when using the genotyping results with respect to the SNP (rs1799998), it is possible to assess that the risk of developing hypertension is highest for the TT genotype, followed by the CT genotype and CC genotype in this order. Alternatively, with respect to the SNP (rs1799998), those with the CC genotype or CT genotype may be assessed as a low risk group whereas those with the TT genotype may be assessed as a high risk group.

In addition, the risk of developing hypertension can be assessed more accurately by using a combination of the genetic markers of the present invention rather than using them individually. For example, those with the TT genotype with respect to the SNP (rs11105378) and the CC genotype or CT genotype with respect to the SNP (rs1799998) may be assessed as a low risk group; whereas those with the TC genotype or CC genotype with respect to the SNP (rs11105378) and the TT genotype with respect to the SNP (rs1799998) may be assessed as a high risk group. In addition, those with the GG genotype or AG genotype with respect to the SNP (rs2681472) and the CC genotype or CT genotype with respect to the SNP (rs1799998) may be assessed as a low risk group; whereas those with the AA genotype with respect to the SNP (rs2681472) and the TT genotype with respect to the SNP (rs1799998) may be assessed as a high risk group. Further, those with the AA genotype with respect to the SNP (rs1401982) and the CC genotype or CT genotype with respect to the SNP (rs1799998) may be assessed as a low risk group; whereas those with the AG genotype or GG genotype with respect to the SNP (rs1401982) and the TT genotype with respect to the SNP (rs1799998) may be assessed as a high risk group. Furthermore, those with the GG genotype with respect to the SNP (rs11105364) and the CC genotype or CT genotype with respect to the SNP (rs1799998) may be assessed as a low risk group; whereas those with the TT genotype or TG genotype with respect to the SNP (rs11105364) and the TT genotype with respect to the SNP (rs1799998) may be assessed as a high risk group.

Alternatively, the genetic markers of the present invention can also be used in combination with other genetic markers for hypertension. There are no particular limitations on the genetic markers to be combined with, and a known genetic marker for hypertension may also be combined for use. It is particularly desirable that a genetic marker for hypertension characterized by including a sequence homologous to or complementary to the partial or complete sequence of an AGT gene (encoding Angiotensinogen) containing a SNP (rs699), which is a SNP of the AGT gene, be combined for use.

The AGT gene is a known hypertension-susceptibility gene which includes a plurality of SNPs. The SNP (rs699) is an MIT polymorphism, and as is apparent from the results described later in Example 28, frequency of the T allele is significantly higher than that of the M allele in the high blood pressure group when compared with the normal blood pressure group. Therefore, the SNP (rs699) is useful as a genetic marker for hypertension. Accordingly, for example, with respect to the SNP (rs699), those with the MM genotype or MT genotype may be assessed as a low risk group whereas those with the TT genotype may be assessed as a high risk group. Note that in the present description and the claims, M stands for methionine (Met) and T stands for threonine (Thr). Here, in terms of the base sequences, the MM genotype, MT genotype and TT genotype are polymorphisms that correspond to those represented as the TT genotype, TC genotype and CC genotype, respectively.

Genotyping of the SNP (rs699) can be carried out by using a known SNP genotyping method as is the case for genotyping of the SNP (rs11105378) or genotyping of the SNP (rs1799998). It is preferable that the SNP (rs699) be identified by a SNP genotyping method in which a polynucleotide having a sequence homologous to or complementary to the partial or complete sequence of the AGT gene is used as a primer or a probe. As a polynucleotide which can be used as a primer or probe for detecting the SNP (rs699) in the AGT gene, a polynucleotide which includes any one of the following base sequences (a) to (f) (hereinafter, referred to as “polynucleotide for detecting SNP (rs699)”) is preferable:

(a) a base sequence represented by sequence number 33 or a base sequence which is a partial sequence of the base sequence represented by sequence number 33 containing the SNP (rs699);

(b) a base sequence complementary to the above-mentioned base sequence (a);

(c) a base sequence composed of the above-mentioned base sequence (a) or (b) in which 1 or more bases other than the SNP (rs699) are deleted, substituted, or added, wherein the polynucleotide including the base sequence can be hybridized with the polynucleotide including the above-mentioned base sequence (a) or (b) under stringent conditions;

(d) a base sequence represented by sequence number 34 or a base sequence which is a partial sequence of the base sequence represented by sequence number 34 containing the SNP (rs699);

(e) a base sequence complementary to the above-mentioned base sequence (d);

(f) a base sequence composed of the above-mentioned base sequence (d) or (e) in which 1 or more bases other than the SNP (rs699) are deleted, substituted, or added, wherein the polynucleotide including the base sequence can be hybridized with the polynucleotide including the above-mentioned base sequence (d) or (e) under stringent conditions.

Note that the polynucleotide for detecting the SNP (rs699) having any one of the above-mentioned base sequences (a) to (c) is a polynucleotide which may detect the T allele of the SNP (rs699), and the polynucleotide for detecting the SNP (rs699) having any one of the above-mentioned base sequences (d) to (f) is a polynucleotide which may detect the M allele of the SNP (rs699).

For example, based on the results of genotyping of the SNP (rs11105378) and the SNP (rs699), those with the TT genotype with respect to the SNP (rs11105378) and the MM genotype or MT genotype with respect to the SNP (rs699) may be assessed as a low risk group; whereas those with the TC genotype or CC genotype with respect to the SNP (rs11105378) and the TT genotype with respect to the SNP (rs699) may be assessed as a high risk group. In addition, based on the results of genotyping of the SNPs (rs2681472) and (rs699), those with the GG genotype or AG genotype with respect to the SNP (rs2681472) and the MM genotype or MT genotype with respect to the SNP (rs699) may be assessed as a low risk group; whereas those with the AA genotype with respect to the SNP (rs2681472) and the TT genotype with respect to the SNP (rs699) may be assessed as a high risk group. Further, based on the results of genotyping of the SNP (rs1401982) and the SNP (rs699), those with the AA genotype with respect to the SNP (rs1401982) and the MM genotype or MT genotype with respect to the SNP (rs699) may be assessed as a low risk group; whereas those with the AG genotype or GG genotype with respect to the SNP (rs1401982) and the TT genotype with respect to the SNP (rs699) may be assessed as a high risk group. Furthermore, based on the results of genotyping of the SNP (rs11105364) and the SNP (rs699), those with the GG genotype with respect to the SNP (rs11105364) and the MM genotype or MT genotype with respect to the SNP (rs699) may be assessed as a low risk group; whereas those with the TT genotype or TG genotype with respect to the SNP (rs11105364) and the TT genotype with respect to the SNP (rs699) may be assessed as a high risk group. Alternatively, based on the results of genotyping of the SNP (rs1799998) and the SNP (rs699), those with the CC genotype or CT genotype with respect to the SNP (rs1799998) and the MM genotype or MT genotype with respect to the SNP (rs699) may be assessed as a low risk group; whereas those with the TT genotype with respect to the SNP (rs1799998) and the TT genotype with respect to the SNP (rs699) may be assessed as a high risk group.

Moreover, by combining three genetic markers, i.e., the genetic marker for hypertension according to the first aspect of the present invention, the genetic marker for hypertension according to the fifth aspect of the present invention, and the genetic marker for hypertension composed of the AGT gene containing a SNP (rs699), a highly reliable risk assessment can be achieved. For example, those with the TT genotype with respect to the SNP (rs11105378), the CC genotype or CT genotype with respect to the SNP (rs1799998) and the MM genotype or MT genotype with respect to the SNP (rs699) may be assessed as a low risk group; whereas those with the TC genotype or CC genotype with respect to the SNP (rs11105378), the TT genotype with respect to the SNP (rs1799998) and the TT genotype with respect to the SNP (rs699) may be assessed as a high risk group. In addition, those with the GG genotype or AG genotype with respect to the SNP (rs2681472), the CC genotype or CT genotype with respect to the SNP (rs1799998) and the MM genotype or MT genotype with respect to the SNP (rs699) may be assessed as a low risk group; whereas those with the AA genotype with respect to the SNP (rs2681472), the TT genotype with respect to the SNP (rs1799998) and the TT genotype with respect to the SNP (rs699) may be assessed as a high risk group. Further, those with the AA genotype with respect to the SNP (rs1401982), the CC genotype or CT genotype with respect to the SNP (rs1799998) and the MM genotype or MT genotype with respect to the SNP (rs699) may be assessed as a low risk group; whereas those with the AG genotype or GG genotype with respect to the SNP (rs1401982), the TT genotype with respect to the SNP (rs1799998) and the TT genotype with respect to the SNP (rs699) may be assessed as a high risk group.

In addition, by combining these three types of genetic markers and based on the number of high risk polymorphisms, the number of low risk polymorphisms, or the like, a more detailed risk assessment can also be achieved. For example, it is possible to assess that the more the number of high risk polymorphisms, the higher the risk of developing hypertension. In addition, it is possible to assess that the less the number of low risk polymorphisms, the higher the risk of developing hypertension. Alternatively, it is also possible to assess that the less the number of high risk polymorphisms, the lower the risk of developing hypertension, and it is also possible to assess that the more the number of low risk polymorphisms, the lower the risk of developing hypertension.

More specifically, by classifying the CC genotype with respect to the SNP (rs11105378), the TT genotype with respect to the SNP (rs1799998) and the TT genotype with respect to the SNP (rs699) as high risk polymorphisms, it is possible to assess that the risk of developing hypertension is highest when the number of these high risk polymorphisms present is 3, followed by the cases where the number of these high risk polymorphisms present is 2, 1 and 0 in this order. In addition, by classifying the AA genotype with respect to the SNP (rs2681472), the TT genotype with respect to the SNP (rs1799998) and the TT genotype with respect to the SNP (rs699) as high risk polymorphisms, it is possible to assess that the risk of developing hypertension is highest when the number of these high risk polymorphisms present is 3, followed by the cases where the number of these high risk polymorphisms present is 2, 1 and 0 in this order. Moreover, by classifying the GG genotype with respect to the SNP (rs1401982), the TT genotype with respect to the SNP (rs1799998) and the TT genotype with respect to the SNP (rs699) as high risk polymorphisms, it is possible to assess that the risk of developing hypertension is highest when the number of these high risk polymorphisms present is 3, followed by the cases where the number of these high risk polymorphisms present is 2, 1 and 0 in this order.

In addition, by classifying the TT genotype with respect to the SNP (rs11105378), the CC genotype with respect to the SNP (rs1799998) and the MM genotype with respect to the SNP (rs699) as low risk polymorphisms, it is possible to assess that the risk of developing hypertension is highest when the number of these low risk polymorphisms present is 0, followed by the cases where the number of these low risk polymorphisms present is 1, 2 and 3 in this order. In addition, by classifying the GG genotype with respect to the SNP (rs2681472), the CC genotype with respect to the SNP (rs1799998) and the MM genotype with respect to the SNP (rs699) as low risk polymorphisms, it is possible to assess that the risk of developing hypertension is highest when the number of these low risk polymorphisms present is 0, followed by the cases where the number of these low risk polymorphisms present is 1, 2 and 3 in this order. Further, by classifying the AA genotype with respect to the SNP (rs1401982), the CC genotype with respect to the SNP (rs1799998) and the MM genotype with respect to the SNP (rs699) as low risk polymorphisms, it is possible to assess that the risk of developing hypertension is highest when the number of these low risk polymorphisms present is 0, followed by the cases where the number of these low risk polymorphisms present is 1, 2 and 3 in this order.

Alternatively, for example, by classifying those with the TT genotype with respect to the SNP (rs11105378), the CC genotype or CT genotype with respect to the SNP (rs1799998) and the MM genotype or MT genotype with respect to the SNP (rs699) as a first group; those with the TC genotype or CC genotype with respect to the SNP (rs11105378), the CC genotype or CT genotype with respect to the SNP (rs1799998) and the MM genotype or MT genotype with respect to the SNP (rs699), those with the TT genotype with respect to the SNP (rs11105378), the CC genotype or CT genotype with respect to the SNP (rs1799998) and the TT genotype with respect to the SNP (rs699), or those with the TT genotype with respect to the SNP (rs11105378), the TT genotype with respect to the SNP (rs1799998) and the MM genotype or MT genotype with respect to the SNP (rs699) as a second group; those with the TC genotype or CC genotype with respect to the SNP (rs11105378), the CC genotype or CT genotype with respect to the SNP (rs1799998) and the TT genotype with respect to the SNP (rs699), those with the TC genotype or CC genotype with respect to the SNP (rs11105378), the TT genotype with respect to the SNP (rs1799998) and the MM genotype or MT genotype with respect to the SNP (rs699), or those with the TT genotype with respect to the SNP (rs11105378), the TT genotype with respect to the SNP (rs1799998) and the TT genotype with respect to the SNP (rs699) as a third group; and those with the TC genotype or CC genotype with respect to the SNP (rs11105378), the TT genotype with respect to the SNP (rs1799998) and the TT genotype with respect to the SNP (rs699) as a fourth group; in the aforementioned step (b), it is possible to assess that the risk of developing hypertension is highest for the above-mentioned fourth group, followed by the above-mentioned third group, the above-mentioned second group and the above-mentioned first group in this order.

In addition, by classifying those with the GG genotype or AG genotype with respect to the SNP (rs2681472), the CC genotype or CT genotype with respect to the SNP (rs1799998) and the MM genotype or MT genotype with respect to the SNP (rs699) as a first group; those with the AA genotype with respect to the SNP (rs2681472), the CC genotype or CT genotype with respect to the SNP (rs1799998) and the MM genotype or MT genotype with respect to the SNP (rs699), those with the GG genotype or AG genotype with respect to the SNP (rs2681472), the CC genotype or CT genotype with respect to the SNP (rs1799998) and the TT genotype with respect to the SNP (rs699), or those with the GG genotype or AG genotype with respect to the SNP (rs2681472), the TT genotype with respect to the SNP (rs1799998) and the MM genotype or MT genotype with respect to the SNP (rs699) as a second group; those with the AA genotype with respect to the SNP (rs2681472), the CC genotype or CT genotype with respect to the SNP (rs1799998) and the TT genotype with respect to the SNP (rs699), those with the AA genotype with respect to the SNP (rs2681472), the TT genotype with respect to the SNP (rs1799998) and the MM genotype or MT genotype with respect to the SNP (rs699), or those with the GG genotype or AG genotype with respect to the SNP (rs2681472), the TT genotype with respect to the SNP (rs1799998) and the TT genotype with respect to the SNP (rs699) as a third group; and those with the AA genotype with respect to the SNP (rs2681472), the TT genotype with respect to the SNP (rs1799998) and the TT genotype with respect to the SNP (rs699) as a fourth group; in the aforementioned step (b), it is possible to assess that the risk of developing hypertension is highest for the above-mentioned fourth group, followed by the above-mentioned third group, the above-mentioned second group and the above-mentioned first group in this order.

Further, by classifying those with the AA genotype with respect to the SNP (rs1401982), the CC genotype or CT genotype with respect to the SNP (rs1799998) and the MM genotype or MT genotype with respect to the SNP (rs699) as a first group; those with the AG genotype or GG genotype with respect to the SNP (rs1401982), the CC genotype or CT genotype with respect to the SNP (rs1799998) and the MM genotype or MT genotype with respect to the SNP (rs699), those with the AA genotype with respect to the SNP (rs1401982), the CC genotype or CT genotype with respect to the SNP (rs1799998) and the TT genotype with respect to the SNP (rs699), or those with the AA genotype with respect to the SNP (rs1401982), the TT genotype with respect to the SNP (rs1799998) and the MM genotype or MT genotype with respect to the SNP (rs699) as a second group; those with the AG genotype or GG genotype with respect to the SNP (rs1401982), the CC genotype or CT genotype with respect to the SNP (rs1799998) and the TT genotype with respect to the SNP (rs699), those with the AG genotype or GG genotype with respect to the SNP (rs1401982), the TT genotype with respect to the SNP (rs1799998) and the MM genotype or MT genotype with respect to the SNP (rs699), or those with the AA genotype with respect to the SNP (rs1401982), the TT genotype with respect to the SNP (rs1799998) and the TT genotype with respect to the SNP (rs699) as a third group; and those with the AG genotype or GG genotype with respect to the SNP (rs1401982), the TT genotype with respect to the SNP (rs1799998) and the TT genotype with respect to the SNP (rs699) as a fourth group; in the aforementioned step (b), it is possible to assess that the risk of developing hypertension is highest for the above-mentioned fourth group, followed by the above-mentioned third group, the above-mentioned second group and the above-mentioned first group in this order.

Alternatively, an assessment on the risk for developing hypertension in the step (b) may be made by combining the genotyping results obtained in the step (a) with one or more risk factors for high blood pressure other than the above-mentioned genetic markers. Examples of the risk factors of a human individual other than the above-mentioned genetic markers include sex, age, body mass index (BMI), the presence of cerebrovascular disease, the presence of cardiac disease, smoking habit, amount of alcohol consumption, total cholesterol, high-density lipoprotein (HDL) cholesterol, neutral fat, and fasting blood sugar.

In those cases where the SNP genotyping in the step (a) is carried out using a microarray method, it is preferable to use the microarray for assessing the risk of developing hypertension according to the eighth aspect of the present invention in which at least one polynucleotide selected from the group consisting of the polynucleotide for detecting the SNP (rs11105378), the polynucleotide for detecting the SNP (rs2681472), the polynucleotide for detecting the SNP (rs1401982), the polynucleotide for detecting the SNP (rs11105364) and the polynucleotide for detecting the SNP (rs1799998) is fixed onto a solid support. As the microarray for assessing the risk of developing hypertension according to the eighth aspect of the present invention, it is preferable that the microarray be one in which at least one polynucleotide selected from the group consisting of the polynucleotide for detecting the SNP (rs11105378), the polynucleotide for detecting the SNP (rs2681472) and the polynucleotide for detecting the SNP (rs1401982), as well as the polynucleotide for detecting the SNP (rs1799998) are fixed onto a solid support; and it is more preferable that the microarray be one in which the polynucleotide for detecting the SNP (rs11105378), the polynucleotide for detecting the SNP (rs2681472), the polynucleotide for detecting the SNP (rs1401982) and the polynucleotide for detecting the SNP (rs1799998) are all fixed onto a solid support.

In addition, as the microarray for assessing the risk of developing hypertension according to the eighth aspect of the present invention, it is preferable that the microarray be one in which at least one polynucleotide selected from the group consisting of the polynucleotide for detecting the SNP (rs11105378), the polynucleotide for detecting the SNP (rs2681472), the polynucleotide for detecting the SNP (rs1401982), the polynucleotide for detecting the SNP (rs11105364), the polynucleotide for detecting the SNP (rs1799998) and the polynucleotide for detecting the SNP (rs699) is fixed onto a solid support; and it is more preferable that the microarray be one in which at least one polynucleotide selected from the group consisting of the polynucleotide for detecting the SNP (rs11105378), the polynucleotide for detecting the SNP (rs2681472), the polynucleotide for detecting the SNP (rs1401982) and the polynucleotide for detecting the SNP (rs1799998), as well as the polynucleotide for detecting the SNP (rs699) are fixed onto a solid support. It is still more preferable that the microarray be one in which at least one polynucleotide selected from the group consisting of the polynucleotide for detecting the SNP (rs11105378), the polynucleotide for detecting the SNP (rs2681472) and the polynucleotide for detecting the SNP (rs1401982), as well as the polynucleotide for detecting the SNP (rs1799998) and the polynucleotide for detecting the SNP (rs699) are fixed onto a solid support; and it is particularly desirable that the microarray be one in which the polynucleotide for detecting the SNP (rs11105378), the polynucleotide for detecting the SNP (rs2681472), the polynucleotide for detecting the SNP (rs1401982), the polynucleotide for detecting the SNP (rs1799998) and the polynucleotide for detecting the SNP (rs699) are all fixed onto a solid support.

Note that in the present invention, the term “microarray” refers to a detection device in which polynucleotides serving as probes are fixed onto a solid support so as to specify the position of each probe, and the support itself in which probes (polynucleotides) are solidified may be in a dispersive state so long as the probes can be fixed onto a two dimensional solid support so as to specify the position of each probe at the time of detection.

In addition, by making the polynucleotides for assessing the risk of developing hypertension and the like which are used for the SNP genotyping in the step (a) into kit, the SNP genotyping in the step (a) can be carried out even more easily. For example, as a SNP genotyping kit used for genotyping at least one SNP selected from the group consisting of the SNP (rs11105378), the SNP (rs2681472), the SNP (rs1401982), the SNP (rs11105364) and the SNP (rs1799998), it is preferable that the SNP genotyping kit be one, just as the SNP genotyping kit for assessing the risk of developing hypertension according to the ninth aspect of the present invention, which includes at least one selected from the group consisting of the polynucleotide for detecting the SNP (rs11105378), the polynucleotide for detecting the SNP (rs2681472), the polynucleotide for detecting the SNP (rs1401982), the polynucleotide for detecting the SNP (rs11105364), the polynucleotide for detecting the SNP (rs1799998) and a microarray in which at least one polynucleotide selected from the group consisting of the polynucleotide for detecting the SNP (rs11105378), the polynucleotide for detecting the SNP (rs2681472), the polynucleotide for detecting the SNP (rs1401982), the polynucleotide for detecting the SNP (rs11105364) and the polynucleotide for detecting the SNP (rs1799998) is fixed onto a solid support. Alternatively, as a SNP genotyping kit used for genotyping at least one SNP selected from the group consisting of the SNP (rs11105378), the SNP (rs2681472), the SNP (rs1401982) and the SNP (rs1799998), as well as the SNP (rs699), it is preferable that the SNP genotyping kit be one, which includes at least one selected from the group consisting of the polynucleotide for detecting the SNP (rs11105378), the polynucleotide for detecting the SNP (rs2681472), the polynucleotide for detecting the SNP (rs1401982), the polynucleotide for detecting the SNP (rs11105364), the polynucleotide for detecting the SNP (rs1799998), and the polynucleotide for detecting the SNP (rs699).

Alternatively, it is possible to construct a loxP integrated small animal in which an ATP2B1 gene is locally deleted by using a loxP integrated vector for constructing small animals those ATP2B1 genes are locally deleted. The loxP integrated vector is characterized by including a base sequence in which the entire ATP2B1 gene sequence or a portion thereof is sandwiched between loxP sequences. To construct a loxP integrated small animal for constructing a small animal in which an ATP2B1 gene is locally deleted, it is particularly desirable to use a loxP integrated vector which includes a base sequence in which the ATP2B1 gene region containing at least one SNP selected from the group consisting of the SNP (rs11105378), the SNP (rs2681472), the SNP (rs1401982) and the SNP (rs11105364) is sandwiched between loxP sequences for constructing a small animal in which an ATP2B1 gene is locally deleted.

Further, it is also possible to construct a small animal in which an ATP2B1 gene is locally deleted by crossing the loxP integrated small animal for constructing a small animal in which an ATP2B1 gene is locally deleted with a transgenic small animal selectively expressing Cre Recombinase which has an adequate promoter as a promoter for the expression of Cre Recombinase. As the promoter for the expression of Cre Recombinase, it is preferable that the promoter be at least one promoter selected from the group consisting of a Tie-2 promoter, a Tie-1 promoter, an Flk-1 promoter, an SM22 promoter, an SM-MHC promoter, a Wt1 promoter, a P0 promoter, a Pax3 promoter, an αMHC promoter, an Nkx2.5 promoter, a Tbx1 promoter, a tetracycline-inducible promoter, and a CMV enhancer-chicken β-actin promoter.

It is preferable that the small animal with the locally deleted ATP2B1 gene which is constructed in the above-mentioned manner exhibits the symptoms of hypertension. In addition, the small animal with the locally deleted ATP2B1 gene can be used as a test animal for screening calcium antagonists, and can also be used as a small animal model for disorders caused by the genetic polymorphisms and impaired expression concerning the ATP2B1 gene.

### EXAMPLES

As follows is a more detailed description of the present invention based on a series of examples, although the scope of the present invention is in no way limited by these examples.

### Example 1

### Preparation of Nucleic Acids Provided for Genotyping of SNPs

A backward cohort study of 8,924 individuals from the general population was conducted in order to investigate the relationship between SNP genotypes and high blood pressure. These individuals were recruited in Yokohama (1,811 subjects), Shiga (3,730 subjects) and Ehime (3,383 subjects).

First, genomic DNA from each individual was extracted from leucocytes in the peripheral blood collected from the individuals using the QIAamp DNA Blood Kit which was a DNA extraction kit manufactured by QIAGEN GmbH. The extracted genomic DNA was amplified using the GenomiPhi DNA Amplification Kit manufactured by GE Healthcare Japan Corporation. The amplified DNA was diluted 50-fold using the buffer AE manufactured by QIAGEN GmbH to be provided for the genotyping of SNPs.

### Example 2

### Genotyping of SNP (rs11105378) in ATP2B1 Gene

The SNP (rs11105378) in the ATP2B1 gene was analyzed by the TaqMan probe method using the amplified genomic DNA of each subject as a template. More specifically, a reaction solution was prepared by adding 2.5 μL of TaqMan Universal Master Mix (manufactured by Applied Biosystems Inc.), 0.05 μL of the TaqMan Pre-Designed SNP Genotyping Assay (Assay ID; C_{—}32174448_{—}10, manufactured by Applied Biosystems Inc.) specific to each polymorphism of rs11105378, and 0.45 μL of distilled water to 2.0 μL of the DNA solution obtained in Example 1, and was then provided for the extension reaction by a PCR method. The extension reaction was conducted through an initial incubation at 52° C. for 2 minutes and a subsequent incubation at 95° C. for 10 minutes, followed by 60 cycles consisting of heating at 95° C. for 15 seconds and at 60° C. for 1 minute. Following the extension reaction, genotyping of genetic polymorphisms was performed by measuring the fluorescence intensity using the 7900 HT Fast Real-Time PCR System (manufactured by Applied Biosystems Inc.).

### Example 3

### Correlation Between rs11105378 Polymorphism and High Blood Pressure

Correlation between the genotype of SNP identified in Example 2 and the high blood pressure among the general population was analyzed by correlation analysis (association method).

TABLE 1

| | |
|---|---|
| Age (years old) | 57 ± 14 |
| Sex (male/female) | 4616/4308 |
| Body mass index (kg/m²) | 23 ± 3 |
| Systolic blood pressure (mmHg) | 132 ± 21 |
| Diastolic blood pressure (mmHg) | 79 ± 12 |
| Antihypertensive medication (yes/no) | 1684/7240 |
| High blood pressure (yes/no) | 3828/5096 |
| Total cholesterol (mg/dL) | 202 ± 35 |
| HDL cholesterol (mg/dL) | 60 ± 15 |
| Neutral fat (mg/dL) | 117 ± 82 |
| Blood sugar (mg/dL) | 101 ± 27 |
| Amount of alcohol consumption (total) | 0.7 ± 1.0 |
| Smoking habit (yes/no) | 2177/6747 |
| Past history of cardiovascular diseases (yes/no) | 580/8344 |

Table 1 shows the clinical backgrounds of 8,924 individuals subjected to the correlation analysis. These individuals were classified into a high blood pressure group (namely, a hypertension group having a systolic blood pressure of at least 140 mmHg and/or a diastolic blood pressure of at least 90 mmHg, and/or taking an antihypertensive agent) and a normal blood pressure group (namely, normotensive group other than those classified as the high blood pressure group), and allele frequencies of the polymorphisms identified in Example 2 were analyzed. A χ²-test (chi-square test) was employed as a statistical analytical method. The results of the analysis are shown in Table 2.

TABLE 2

| | | | | Results of statistical analysis | | | |
|---|---|---|---|---|---|---|---|
| | Frequency of genetic | | | (upper box: odds ratio, lower box: p-value) | | | |
| | polymorphism | | | Allele | | | |
| | rs11105378 polymorphism | | | frequency | Frequency of genetic polymorphism | | |
| | TT | TC | CC | T/C | TT/TC + CC | TT + TC/CC | TT/TC/CC |
| High blood | 438 | 1719 | 1671 | 1.138 | 1.256 | 1.147 | — |
| pressure group | (11.4) | (44.9) | (43.7) | <0.001 | <0.001 | 0.002 | <0.001 |
| Normal blood | 712 | 2330 | 2054 | | | | |
| pressure group | (14.0) | (45.7) | (40.3) | | | | |

From the results described in Table 2, it became apparent that among the high blood pressure group and the normal blood pressure group, frequencies of each genotype with respect to the rs11105378 polymorphisms identified in Example 2 showed statistically significant difference. More specifically, it became clear from the odds ratio that frequency of the C allele was significantly higher than that of the T allele in the high blood pressure group when compared with the normal blood pressure group.

From the above results, it is evident that the relative risk for developing hypertension associated with the genetic polymorphisms can be assessed by examining the genotypes of SNP (rs11105378).

### Example 4

### Correlation Between rs11105378 Polymorphism and High Blood Pressure Analyzed by Logistic Regression Analysis

Correlation between the rs11105378 polymorphism identified in Example 2 and high blood pressure was analyzed by a regression analysis that included other relevant environmental factors.

The individuals shown in Table 1 were classified into a high blood pressure group and a normal blood pressure group in the same manner as that described in Example 3. A logistic regression analysis was carried out by using the above classification (namely, the high blood pressure group and the normal blood pressure group) as a dependent variable, while using sex, age, body mass index, past history of cardiovascular diseases, smoking habit, amount of alcohol consumption, high-density lipoprotein (HDL) cholesterol, neutral fat, and blood sugar, in addition to the rs11105378 polymorphism identified in Example 2, as independent variables. The results of the analysis are shown in Table 3.

TABLE 3

| | | Significance | Odds | Confidence |
|---|---|---|---|---|
| | | probability | ratio | interval |
| Age (years old) | | <0.001 | 1.081 | 1.076-1.087 |
| Sex (female) | | 0.313 | 0.937 | 0.826-1.063 |
| Body mass index (kg/m²) | | <0.001 | 1.201 | 1.180-1.223 |
| Past history of cardiovascular | | <0.001 | 1.511 | 1.228-1.859 |
| diseases | | | | |
| Smoking habit (yes/no) | | 0.58 | 0.964 | 0.846-1.098 |
| Amount of alcohol consumption | | <0.001 | 1.211 | 1.138-1.288 |
| (total) | | | | |
| HDL cholesterol (mg/dL) | | <0.001 | 1.014 | 1.010-1.017 |
| Neutral fat (mg/dL) | | <0.001 | 1.003 | 1.076-1.087 |
| Blood sugar (mg/dL) | | <0.001 | 1.005 | 1.003-1.007 |
| rs11105378 polymorphism | TT | Control | | |
| | TC | 0.006 | 1.244 | 1.065-1.452 |
| | CC | <0.001 | 1.404 | 1.201-1.642 |

From the results shown in Table 3, it became apparent that even after adjusted to other environmental factors such as sex, age, body mass index, past history of cardiovascular diseases, smoking habit, amount of alcohol consumption, HDL cholesterol, neutral fat, and blood sugar, the rs11105378 polymorphism identified in Example 2 remained an independent risk factor for high blood pressure. In addition, the relative risk thereof (represented by odds ratio) was 1.244 fold for the TC genotype and 1.404 fold for the CC genotype, when compared to the TT genotype.

From the above results, it is evident that the relative risk for developing hypertension can be assessed, even after adjusted to the effects of other environmental factors, by examining the rs11105378 polymorphism. Moreover, it is also apparent from the above results that with respect to the SNP (rs11105378), those with the TT genotype may be classified into a low risk group whereas those with the TC genotype or CC genotype may be classified into a high risk group.

### Example 5

### Rough Calculation of Mean Blood Pressure for Each rs11105378 Polymorphism

Mean values of systolic blood pressure and diastolic blood pressure were roughly determined for each polymorphism identified in Example 2, and the differences therebetween were statistically analyzed by one-way analysis of variance. The results of the analysis are shown in Table 4.

TABLE 4

| | rs11105378 polymorphism | | | |
|---|---|---|---|---|
| | TT | TC | CC | |
| | (1150 | (4049 | (3725 | |
| | subjects) | subjects) | subjects) | p-value |
| Systolic blood pressure | 130 ± 20 | 132 ± 20 | 133 ± 21 | <0.001 |
| (mmHg) | | | | |
| Diastolic blood pressure | 78 ± 11 | 79 ± 12 | 80 ± 12 | <0.001 |
| (mmHg) | | | | |

From the results shown in Table 4, it became apparent that mean values of systolic blood pressure and diastolic blood pressure determined roughly for each rs11105378 polymorphism all differed statistically significantly.

From the above results, it is evident that the degree of blood pressure elevation for each genetic polymorphism may be estimated by examining the rs11105378 polymorphism.

### Example 6

### Correlation Between rs11105378 Polymorphism and Blood Pressure Analyzed by Multiple Regression Analysis

Correlation between the rs11105378 polymorphism identified in Example 2 and blood pressure was analyzed by a regression analysis that included other relevant environmental factors.

A multiple regression analysis was carried out by using systolic blood pressure or diastolic blood pressure as a dependent variable, while using sex, age, body mass index, past history of cardiovascular diseases, smoking habit, amount of alcohol consumption, use of antihypertensive medication, HDL cholesterol, neutral fat, blood sugar, and cohort variables, in addition to the rs11105378 polymorphism identified in Example 2, as independent variables. The results of the analysis are shown in Table 5.

TABLE 5

| | | Systolic blood pressure | | | Diastolic blood pressure | | |
|---|---|---|---|---|---|---|---|
| | | Unnormalized | Normalized | | Unnormalized | Normalized | |
| | | coefficient | coefficient | p-value | coefficient | coefficient | p-value |
| Age (years old) | | 0.505 | 0.333 | <0.001 | 0.168 | 0.192 | <0.001 |
| Sex (female) | | −2.394 | −0.058 | <0.001 | −3.520 | −0.148 | <0.001 |
| Body mass index (kg/m²) | | 1.283 | 0.193 | <0.001 | 0.905 | 0.235 | <0.001 |
| Past history of cardiovascular | | −3.335 | −0.040 | <0.001 | −2.377 | −0.049 | <0.001 |
| diseases (yes/no) | | | | | | | |
| Smoking habit (yes/no) | | −0.232 | −0.005 | 0.630 | −0.733 | −0.026 | 0.013 |
| Amount of alcohol consumption | | 1.434 | 0.068 | <0.001 | 1.119 | 0.092 | <0.001 |
| (total) | | | | | | | |
| Antihypertensive medication | | 10.591 | 0.202 | <0.001 | 3.987 | 0.131 | <0.001 |
| (yes/no) | | | | | | | |
| HDL cholesterol (mg/dL) | | 0.087 | 0.066 | <0.001 | 0.075 | 0.097 | <0.001 |
| Neutral fat (mg/dL) | | 0.019 | 0.076 | <0.001 | 0.016 | 0.108 | <0.001 |
| Blood sugar (mg/dL) | | 0.038 | 0.050 | <0.001 | 0.000 | −0.001 | 0.947 |
| ra11105378 polymorphism | TT | Control | | | Control | | |
| | TC | 1.889 | 0.046 | 0.001 | 0.963 | 0.040 | 0.006 |
| | CC | 2.707 | 0.065 | <0.001 | 1.462 | 0.061 | <0.001 |

From the results shown in Table 5, it became apparent that even after adjusted to other environmental factors such as sex, age, body mass index, past history of cardiovascular diseases, smoking habit, amount of alcohol consumption, use of antihypertensive medication, HDL cholesterol, neutral fat, blood sugar, and cohort variables, the rs11105378 polymorphism identified in Example 2 remained an independent risk factor for systolic blood pressure or diastolic blood pressure.

From the above results, it is evident that the degree of blood pressure elevation for each genetic polymorphism may be estimated, even after adjusted to the effects of other environmental factors, by examining the rs11105378 polymorphism.

### Example 7

### Calculation of Adjusted Mean Blood Pressure for Each rs11105378 Polymorphism

From the regression analysis described in Example 6 which analyzed correlation between the rs11105378 polymorphism identified in Example 2 and blood pressure, mean values of systolic blood pressure and diastolic blood pressure for each rs11105378 polymorphism were calculated after adjusted to sex, age, body mass index, past history of cardiovascular diseases, smoking habit, amount of alcohol consumption, use of antihypertensive medication, HDL cholesterol, neutral fat, blood sugar, and cohort variables (i.e., adjusted mean blood pressure). The results of the analysis are shown in Table 6.

TABLE 6

| | rs11105378 polymorphism | | | |
|---|---|---|---|---|
| | (mean ± standard error) | | | |
| | TT | TC | CC | |
| | (1150) | (4049) | (3725) | p-value |
| Systolic blood pressure | 130 ± 0.5 | 132 ± 0.3 | 133 ± 0.3 | <0.001 |
| (mmHg) | | | | |
| Diastolic blood pressure | 78 ± 0.3 | 79 ± 0.2 | 80 ± 0.2 | <0.001 |
| (mmHg) | | | | |

From the results shown in Table 6, it became apparent that adjusted mean values of systolic blood pressure and diastolic blood pressure determined for each rs11105378 polymorphism differed statistically significantly.

From the above results, it is evident that the degree of blood pressure elevation for each genetic polymorphism may be estimated, even after adjusted to the effects of other environmental factors, by examining the rs11105378 polymorphism.

### Example 8

### Genotyping of SNP (rs2681472) in ATP2B1 Gene

A backward cohort study of 9,452 individuals from the general population was conducted in order to investigate the relationship between SNP genotypes and high blood pressure. These individuals were recruited in Yokohama (1,871 subjects), Shiga (4,021 subjects) and Ehime (3,560 subjects).

First, genomic DNA from each individual was extracted from leucocytes in the peripheral blood collected from the individuals and was then amplified in the same manner as that described in Example 1, thereby obtaining a DNA solution to be provided for the genotyping of SNPs.

The SNP (rs2681472) in the ATP2B1 gene was analyzed by the TaqMan probe method using the amplified genomic DNA of each subject as a template which was obtained in the above-mentioned manner. More specifically, genotyping of genetic polymorphisms was performed in the same manner as that described in Example 2 except that the TaqMan Pre-Designed SNP Genotyping Assay (Assay ID; C_{—}16057071_{—}10, manufactured by Applied Biosystems Inc.) specific to each polymorphism of rs2681472 was used instead of the TaqMan Pre-Designed SNP Genotyping Assay specific to each polymorphism of rs11105378.

### Example 9

### Correlation Between rs2681472 Polymorphism and High Blood Pressure

Correlation between the genotype of SNP identified in Example 8 and the high blood pressure among the individuals was analyzed by correlation analysis (association method).

TABLE 7

| | |
|---|---|
| Age (years old) | 57 ± 14 |
| Sex (male/female) | 4859/4593 |
| Body mass index (kg/m²) | 23 ± 3 |
| Systolic blood pressure (mmHg) | 132 ± 21 |
| Diastolic blood pressure (mmHg) | 79 ± 12 |
| Antihypertensive medication (yes/no) | 1780/7672 |
| High blood pressure (yes/no) | 4067/5385 |
| Total cholesterol (mg/dL) | 202 ± 35 |
| HDL cholesterol (mg/dL) | 60 ± 15 |
| Neutral fat (mg/dL) | 117 ± 83 |
| Blood sugar (mg/dL) | 101 ± 27 |
| Amount of alcohol consumption (total) | 0.7 ± 1.0 |
| Smoking habit (yes/no) | 2293/7159 |
| Past history of cardiovascular diseases (yes/no) | 621/8831 |

Table 7 shows the clinical backgrounds of 9,452 individuals subjected to the correlation analysis. These individuals were classified into a high blood pressure group (namely, a hypertension group having a systolic blood pressure of at least 140 mmHg and/or a diastolic blood pressure of at least 90 mmHg, and/or taking an antihypertensive agent) and a normal blood pressure group (namely, normotensive group other than those classified as the high blood pressure group), and allele frequencies of the polymorphisms identified in Example 8 were analyzed. A χ²-test was employed as a statistical analytical method. The results of the analysis are shown in Table 8.

TABLE 8

| | | | | Results of statistical analysis | | | |
|---|---|---|---|---|---|---|---|
| | Frequency of genetic | | | (upper box: odds ratio, lower box: p-value) | | | |
| | polymorphism | | | Allele | Frequency of genetic polymorphism | | |
| | rs2681472 polymorphism | | | frequency | AA/ | AA + AG/ | |
| | AA | AG | GG | A/G | AG + GG | GG | AA/AG/GG |
| High blood | 1703 | 1858 | 506 | 1.115 | 1.129 | 1.199 | — |
| pressure group | (41.9) | (45.7) | (12.4) | <0.001 | 0.004 | 0.003 | 0.002 |
| Normal blood | 2098 | 2503 | 784 | | | | |
| pressure group | (39.0) | (46.5) | (14.6) | | | | |

From the results described in Table 8, it became apparent that among the high blood pressure group and the normal blood pressure group, frequencies of each genotype with respect to the rs2681472 polymorphism identified in Example 8 showed statistically significant difference. More specifically, it became clear from the odds ratio that frequency of the A allele was significantly higher than that of the G allele in the high blood pressure group when compared with the normal blood pressure group.

From the above results, it is evident that the relative risk for developing hypertension associated with the genetic polymorphisms can be assessed by examining the genotypes of SNP (rs2681472).

### Example 10

### Correlation Between rs2681472 Polymorphism and High Blood Pressure Analyzed by Logistic Regression Analysis

Correlation between the rs2681472 polymorphism identified in Example 8 and high blood pressure was analyzed by a regression analysis that included other relevant environmental factors.

The individuals shown in Table 7 were classified into a high blood pressure group and a normal blood pressure group in the same manner as that described in Example 9. A logistic regression analysis was carried out by using the above classification (namely, the high blood pressure group and the normal blood pressure group) as a dependent variable, while using sex, age, body mass index, past history of cardiovascular diseases, smoking habit, amount of alcohol consumption, HDL cholesterol, neutral fat, blood sugar and cohort variables, in addition to the rs2681472 polymorphism identified in Example 8, as independent variables. The results of the analysis are shown in Table 9.

TABLE 9

| | | Significance | Odds | Confidence |
|---|---|---|---|---|
| | | probability | ratio | interval |
| Age (years old) | | <0.001 | 1.081 | 1.076-1.086 |
| Sex (female) | | 0.183 | 0.920 | 0.814-1.040 |
| Body mass index (kg/m²) | | <0.001 | 1.203 | 1.182-1.224 |
| Past history of cardiovascular | | <0.001 | 1.513 | 1.239-1.848 |
| diseases | | | | |
| Smoking habit (yes/no) | | 0.633 | 0.970 | 0.855-1.100 |
| Amount of alcohol consumption | | <0.001 | 1.217 | 1.145-1.293 |
| (total) | | | | |
| HDL cholesterol (mg/dL) | | <0.001 | 1.013 | 1.010-1.017 |
| Neutral fat (mg/dL) | | <0.001 | 1.003 | 1.002-1.004 |
| Blood sugar (mg/dL) | | <0.001 | 1.005 | 1.003-1.007 |
| rs2681472 polymorphism | GG | Control | | |
| | AG | 0.028 | 1.179 | 1.018-1.364 |
| | AA | <0.001 | 1.348 | 1.162-1.564 |

From the results shown in Table 9, it became apparent that even after adjusted to other environmental factors such as sex, age, body mass index, past history of cardiovascular diseases, smoking habit, amount of alcohol consumption, HDL cholesterol, neutral fat, blood sugar and cohort variables, the rs2681472 polymorphism identified in Example 8 remained an independent risk factor for high blood pressure. In addition, the relative risk thereof (represented by odds ratio) was 1.179 fold for the AG genotype and 1.348 fold for the AA genotype, when compared to the GG genotype.

From the above results, it is evident that the relative risk for developing hypertension can be assessed, even after adjusted to the effects of other environmental factors, by examining the rs2681472 polymorphism. Moreover, it is also apparent from the above results that with respect to the SNP (rs2681472), those with the GG genotype or AG genotype may be classified into a low risk group whereas those with the AA genotype may be classified into a high risk group.

### Example 11

### Rough Calculation of Mean Blood Pressure for Each rs2681472 Polymorphism

Mean values of systolic blood pressure and diastolic blood pressure were roughly calculated for each polymorphism identified in Example 8, and the differences therebetween were statistically analyzed by one-way analysis of variance. The results of the analysis are shown in Table 10.

TABLE 10

| | rs2681472 polymorphism | | | |
|---|---|---|---|---|
| | GG | AG | AA | |
| | (1290) | (4361) | (3801) | p-value |
| Systolic blood pressure | 130 ± 20 | 132 ± 20 | 133 ± 21 | <0.001 |
| (mmHg) | | | | |
| Diastolic blood pressure | 78 ± 11 | 79 ± 12 | 80 ± 12 | <0.001 |
| (mmHg) | | | | |

From the results shown in Table 10, it became apparent that mean values of systolic blood pressure and diastolic blood pressure determined roughly for each rs2681472 polymorphism all differed statistically significantly.

From the above results, it is evident that the degree of blood pressure elevation for each genetic polymorphism may be estimated by examining the rs2681472 polymorphism.

### Example 12

### Correlation Between rs2681472 Polymorphism and Blood Pressure Analyzed by Multiple Regression Analysis

Correlation between the rs2681472 polymorphism identified in Example 8 and blood pressure was analyzed by a regression analysis that included other relevant environmental factors.

A multiple regression analysis was carried out by using systolic blood pressure or diastolic blood pressure as a dependent variable, while using sex, age, body mass index, past history of cardiovascular diseases, smoking habit, amount of alcohol consumption, use of antihypertensive medication, HDL cholesterol, neutral fat, blood sugar, and cohort variables, in addition to the rs2681472 polymorphism identified in Example 8, as independent variables. The results of the analysis are shown in Table 11.

TABLE 11

| | | Systolic blood pressure | | | Diastolic blood pressure | | |
|---|---|---|---|---|---|---|---|
| | | Unnormalized | Normalized | | Unnormalized | Normalized | |
| | | coefficient | coefficient | p-value | coefficient | coefficient | p-value |
| Age (years old) | | 0.509 | 0.335 | <0.001 | 0.169 | 0.192 | <0.001 |
| Sex (female) | | −2.606 | −0.063 | <0.001 | −3.647 | −0.153 | <0.001 |
| Body mass index (kg/m²) | | 1.299 | 0.196 | <0.001 | 0.914 | 0.238 | <0.001 |
| Past history of cardiovascular | | −3.303 | −0.040 | <0.001 | −2.312 | −0.048 | <0.001 |
| diseases (yes/no) | | | | | | | |
| Smoking habit (yes/no) | | −0.431 | −0.009 | 0.356 | −0.905 | −0.033 | 0.002 |
| Amount of alcohol consumption | | 1.446 | 0.068 | <0.001 | 1.159 | 0.095 | <0.001 |
| (total) | | | | | | | |
| Antihypertensive medication | | 10.608 | 0.202 | <0.001 | 3.956 | 0.130 | <0.001 |
| (yes/no) | | | | | | | |
| HDL cholesterol (mg/dL) | | 0.084 | 0.063 | <0.001 | 0.070 | 0.091 | <0.001 |
| Neutral fat (mg/dL) | | 0.019 | 0.076 | <0.001 | 0.016 | 0.107 | <0.001 |
| Blood sugar (mg/dL) | | 0.038 | 0.050 | <0.001 | 0.000 | −0.001 | 0.956 |
| rs2681472 polymorphism | GG | Control | | | Control | | |
| | AG | 1.988 | 0.048 | <0.001 | 0.905 | 0.038 | 0.006 |
| | AA | 2.885 | 0.069 | <0.001 | 1.465 | 0.060 | <0.001 |

From the results shown in Table 11, it became apparent that even after adjusted to other environmental factors such as sex, age, body mass index, past history of cardiovascular diseases, smoking habit, amount of alcohol consumption, use of antihypertensive medication, HDL cholesterol, neutral fat, blood sugar, and cohort variables, the rs2681472 polymorphism identified in Example 8 remained an independent risk factor for systolic blood pressure or diastolic blood pressure.

From the above results, it is evident that the degree of blood pressure elevation for each genetic polymorphism may be estimated, even after adjusted to the effects of other environmental factors, by examining the rs2681472 polymorphism.

### Example 13

### Calculation of Adjusted Mean Blood Pressure for Each rs2681472 Polymorphism

From the regression analysis described in Example 12 which analyzed correlation between the rs2681472 polymorphism identified in Example 8 and blood pressure, mean values of systolic blood pressure and diastolic blood pressure for each rs2681472 polymorphism were calculated after adjusted to sex, age, body mass index, past history of cardiovascular diseases, smoking habit, amount of alcohol consumption, use of antihypertensive medication, HDL cholesterol, neutral fat, blood sugar, and cohort variables (i.e., adjusted mean blood pressure). The results of the analysis are shown in Table 12.

TABLE 12

| | rs2681472 polymorphism | | | |
|---|---|---|---|---|
| | (mean ± standard error) | | | |
| | GG | AG | AA | |
| | (1290) | (4361) | (3801) | p-value |
| Systolic blood pressure | 130 ± 0.5 | 132 ± 0.3 | 133 ± 0.3 | <0.001 |
| (mmHg) | | | | |
| Diastolic blood pressure | 78 ± 0.3 | 79 ± 0.2 | 80 ± 0.2 | <0.001 |
| (mmHg) | | | | |

From the results shown in Table 12, it became apparent that adjusted mean values of systolic blood pressure and diastolic blood pressure determined for each rs2681472 polymorphism differed statistically significantly.

From the above results, it is evident that the degree of blood pressure elevation for each genetic polymorphism may be estimated, even after adjusted to the effects of other environmental factors, by examining the rs2681472 polymorphism.

### Example 14

### Genotyping of SNP (rs1401982) in ATP2B1 Gene

A backward cohort study of 9,388 individuals from the general population was conducted in order to investigate the relationship between SNP genotypes and high blood pressure. These individuals were recruited in Yokohama (1,869 subjects), Shiga (3,950 subjects) and Ehime (3,569 subjects).

First, genomic DNA from each individual was extracted from leucocytes in the peripheral blood collected from the individuals and was then amplified in the same manner as that described in Example 1, thereby obtaining a DNA solution to be provided for the genotyping of SNPs.

The SNP (rs1401982) in the ATP2B1 gene was analyzed by the TaqMan probe method using the amplified genomic DNA of each subject as a template which was obtained in the above-mentioned manner. More specifically, genotyping of genetic polymorphisms was performed in the same manner as that described in Example 2 except that the TaqMan Pre-Designed SNP Genotyping Assay (Assay ID; C_{—}2775503_{—}10, manufactured by Applied Biosystems Inc.) specific to each polymorphism of rs1401982 was used instead of the TaqMan Pre-Designed SNP Genotyping Assay specific to each polymorphism of rs11105378.

### Example 15

### Correlation Between rs1401982 Polymorphism and High Blood Pressure

Correlation between the genotype of SNP identified in Example 14 and the high blood pressure among the individuals was analyzed by correlation analysis (association method).

TABLE 13

| | |
|---|---|
| Age (years old) | 57 ± 14 |
| Sex (male/female) | 4839/4549 |
| Body mass index (kg/m²) | 23 ± 3 |
| Systolic blood pressure (mmHg) | 132 ± 21 |
| Diastolic blood pressure (mmHg) | 79 ± 12 |
| Antihypertensive medication (yes/no) | 1769/7619 |
| High blood pressure (yes/no) | 4029/5359 |
| Total cholesterol (mg/dL) | 202 ± 35 |
| HDL cholesterol (mg/dL) | 60 ± 15 |
| Neutral fat (mg/dL) | 117 ± 83 |
| Blood sugar (mg/dL) | 101 ± 27 |
| Amount of alcohol consumption (total) | 0.7 ± 1.0 |
| Smoking habit (yes/no) | 2284/7104 |
| Past history of cardiovascular diseases (yes/no) | 609/8779 |

Table 13 shows the clinical backgrounds of 9,388 individuals subjected to the correlation analysis. These individuals were classified into a high blood pressure group (namely, a hypertension group having a systolic blood pressure of at least 140 mmHg and/or a diastolic blood pressure of at least 90 mmHg, and/or taking an antihypertensive agent) and a normal blood pressure group (namely, normotensive group other than those classified as the high blood pressure group), and allele frequencies of the polymorphisms identified in Example 14 were analyzed. A χ²-test was employed as a statistical analytical method. The results of the analysis are shown in Table 14.

TABLE 14

| | | | | Results of statistical analysis | | | |
|---|---|---|---|---|---|---|---|
| | Frequency of genetic | | | (upper box: odds ratio, lower box: p-value) | | | |
| | polymorphism | | | Allele | Frequency of genetic polymorphism | | |
| | rs1401982 polymorphism | | | frequency | AA/ | AA + AG/ | |
| | AA | AG | GG | A/G | AG + GG | GG | AA/AG/GG |
| High blood | 1654 | 1851 | 524 | 1.110 | 1.133 | 1.170 | — |
| pressure group | (41.1) | (45.9) | (13.0) | 0.001 | 0.003 | 0.009 | 0.003 |
| Normal blood | 2040 | 2521 | 798 | | | | |
| pressure group | (38.1) | (47.0) | (14.9) | | | | |

From the results described in Table 14, it became apparent that among the high blood pressure group and the normal blood pressure group, frequencies of each genotype with respect to the rs1401982 polymorphisms identified in Example 14 showed statistically significant difference. More specifically, it became clear from the odds ratio that frequency of the G allele was significantly higher than that of the A allele in the high blood pressure group when compared with the normal blood pressure group.

From the above results, it is evident that the relative risk for developing hypertension associated with the genetic polymorphisms can be assessed by examining the genotypes of SNP (rs1401982).

### Example 16

### Correlation Between rs1401982 Polymorphism and High Blood Pressure Analyzed by Logistic Regression Analysis

Correlation between the rs1401982 polymorphism identified in Example 14 and high blood pressure was analyzed by a regression analysis that included other relevant environmental factors.

The individuals shown in Table 13 were classified into a high blood pressure group and a normal blood pressure group in the same manner as that described in Example 15. A logistic regression analysis was carried out by using the above classification (namely, the high blood pressure group and the normal blood pressure group) as a dependent variable, while using sex, age, body mass index, past history of cardiovascular diseases, smoking habit, amount of alcohol consumption, HDL cholesterol, neutral fat, blood sugar and cohort variables, in addition to the rs1401982 polymorphism identified in Example 14, as independent variables. The results of the analysis are shown in Table 15.

TABLE 15

| | | Significance | Odds | Confidence |
|---|---|---|---|---|
| | | probability | ratio | interval |
| Age (years old) | | <0.001 | 1.081 | 1.075-1.086 |
| Sex (female) | | 0.178 | 0.919 | 0.812-1.039 |
| Body mass index (kg/m²) | | <0.001 | 1.202 | 1.182-1.224 |
| Past history of cardiovascular | | <0.001 | 1.520 | 1.243-1.858 |
| diseases | | | | |
| Smoking habit (yes/no) | | 0.786 | 0.983 | 0.866-1.115 |
| Amount of alcohol consumption | | <0.001 | 1.213 | 1.141-1.288 |
| (total) | | | | |
| HDL cholesterol (mg/dL) | | <0.001 | 1.014 | 1.010-1.018 |
| Neutral fat (mg/dL) | | <0.001 | 1.003 | 1.002-1.004 |
| Blood sugar (mg/dL) | | <0.001 | 1.005 | 1.003-1.007 |
| rs1401982 polymorphism | AA | Control | | |
| | AG | 0.063 | 1.147 | 0.993-1.326 |
| | GG | <0.001 | 1.317 | 1.136-1.527 |

From the results shown in Table 15, it became apparent that even after adjusted to other environmental factors such as sex, age, body mass index, past history of cardiovascular diseases, smoking habit, amount of alcohol consumption, HDL cholesterol, neutral fat, blood sugar and cohort variables, the rs1401982 polymorphism identified in Example 14 remained an independent risk factor for high blood pressure. In addition, the relative risk thereof (represented by odds ratio) was 1.179 fold for the AG genotype and 1.348 fold for the AA genotype, when compared to the GG genotype.

From the above results, it is evident that the relative risk for developing hypertension can be assessed, even after adjusted to the effects of other environmental factors, by examining the rs1401982 polymorphism. Moreover, it is also apparent from the above results that with respect to the SNP (rs1401982), those with the GG genotype or AG genotype may be classified into a low risk group whereas those with the AA genotype may be classified into a high risk group.

### Example 17

### Rough Calculation of Mean Blood Pressure for Each rs1401982 Polymorphism

Mean values of systolic blood pressure and diastolic blood pressure were roughly calculated for each polymorphism identified in Example 14, and the differences therebetween were statistically analyzed by one-way analysis of variance. The results of the analysis are shown in Table 16.

TABLE 16

| | rs1401982 polymorphism | | | |
|---|---|---|---|---|
| | GG | AG | AA | |
| | (3694) | (4372) | (1322) | p-value |
| Systolic blood pressure | 133 ± 21 | 132 ± 21 | 130 ± 20 | <0.001 |
| (mmHg) | | | | |
| Diastolic blood pressure | 80 ± 12 | 79 ± 12 | 78 ± 11 | <0.001 |
| (mmHg) | | | | |

From the results shown in Table 16, it became apparent that mean values of systolic blood pressure and diastolic blood pressure determined roughly for each rs1401982 polymorphism all differed statistically significantly.

From the above results, it is evident that the degree of blood pressure elevation for each genetic polymorphism may be estimated by examining the rs1401982 polymorphism.

### Example 18

### Correlation Between rs1401982 Polymorphism and Blood Pressure Analyzed by Multiple Regression Analysis

Correlation between the rs1401982 polymorphism identified in Example 14 and blood pressure was analyzed by a regression analysis that included other relevant environmental factors.

A multiple regression analysis was carried out by using systolic blood pressure or diastolic blood pressure as a dependent variable, while using sex, age, body mass index, past history of cardiovascular diseases, smoking habit, amount of alcohol consumption, use of antihypertensive medication, HDL cholesterol, neutral fat, blood sugar, and cohort variables, in addition to the rs1401982 polymorphism identified in Example 14, as independent variables. The results of the analysis are shown in Table 17.

TABLE 17

| | | Systolic blood pressure | | | Diastolic blood pressure | | |
|---|---|---|---|---|---|---|---|
| | | Unnormalized | Normalized | | Unnormalized | Normalized | |
| | | coefficient | coefficient | p-value | coefficient | coefficient | p-value |
| Age (years old) | | 0.505 | 0.332 | <0.001 | 0.167 | 0.190 | <0.001 |
| Sex (female) | | −2.489 | −0.060 | <0.001 | −3.549 | −0.149 | <0.001 |
| Body mass index (kg/m²) | | 1.286 | 0.194 | <0.001 | 0.908 | 0.237 | <0.001 |
| Past history of cardiovascular | | −3.419 | −0.041 | <0.001 | −2.320 | −0.048 | <0.001 |
| diseases (yes/no) | | | | | | | |
| Smoking habit (yes/no) | | −0.257 | −0.005 | 0.584 | −0.788 | −0.028 | 0.006 |
| Amount of alcohol consumption | | 1.458 | 0.069 | <0.001 | 1.167 | 0.095 | <0.001 |
| (total) | | | | | | | |
| Antihypertensive medication | | 10.612 | 0.202 | <0.001 | 4.005 | 0.132 | <0.001 |
| (yes/no) | | | | | | | |
| HDL cholesterol (mg/dL) | | 0.085 | 0.064 | <0.001 | 0.070 | 0.091 | <0.001 |
| Neutral fat (mg/dL) | | 0.019 | 0.077 | <0.001 | 0.016 | 0.107 | <0.001 |
| Blood sugar (mg/dL) | | 0.038 | 0.050 | <0.001 | 0.000 | 0.001 | 0.950 |
| rs1401982 polymorphism | AA | Control | | | Control | | |
| | AG | 1.889 | 0.046 | <0.001 | 0.850 | 0.036 | 0.009 |
| | GG | 2.767 | 0.066 | <0.001 | 1.385 | 0.057 | <0.001 |

From the results shown in Table 17, it became apparent that even after adjusted to other environmental factors such as sex, age, body mass index, past history of cardiovascular diseases, smoking habit, amount of alcohol consumption, use of antihypertensive medication, HDL cholesterol, neutral fat, blood sugar, and cohort variables, the rs1401982 polymorphism identified in Example 14 remained an independent risk factor for systolic blood pressure or diastolic blood pressure.

From the above results, it is evident that the degree of blood pressure elevation for each genetic polymorphism may be estimated, even after adjusted to the effects of other environmental factors, by examining the rs1401982 polymorphism.

### Example 19

### Calculation of Adjusted Mean Blood Pressure for Each rs1401982 Polymorphism

From the regression analysis described in Example 18 which analyzed correlation between the rs1401982 polymorphism identified in Example 14 and blood pressure, mean values of systolic blood pressure and diastolic blood pressure for each rs1401982 polymorphism were calculated after adjusted to sex, age, body mass index, past history of cardiovascular diseases, smoking habit, amount of alcohol consumption, use of antihypertensive medication, HDL cholesterol, neutral fat, blood sugar, and cohort variables (i.e., adjusted mean blood pressure). The results of the analysis are shown in Table 18.

TABLE 18

| | rs1401982 polymorphism | | | |
|---|---|---|---|---|
| | (mean ± standard error) | | | |
| | GG | AG | AA | |
| | (3694) | (4372) | (1322) | p-value |
| Systolic blood pressure | 133 ± 0.3 | 132 ± 0.3 | 130 ± 0.5 | <0.001 |
| (mmHg) | | | | |
| Diastolic blood pressure | 80 ± 0.2 | 79 ± 0.2 | 78 ± 0.3 | <0.001 |
| (mmHg) | | | | |

From the results shown in Table 18, it became apparent that adjusted mean values of systolic blood pressure and diastolic blood pressure determined for each rs1401982 polymorphism differed statistically significantly.

From the above results, it is evident that the degree of blood pressure elevation for each genetic polymorphism may be estimated, even after adjusted to the effects of other environmental factors, by examining the rs1401982 polymorphism.

### Example 20

### Genotyping of SNP (rs11105364) in ATP2B1 Gene

The polymorphic site (rs11105364) in the ATP2B1 gene was analyzed by the TaqMan probe method using the amplified genomic DNA of each subject as a template which was obtained in Example 1. More specifically, a reaction solution was prepared by adding 2.5 μL of TaqMan Universal Master Mix (manufactured by Applied Biosystems Inc.), 0.05 μL of the TaqMan Pre-Designed SNP Genotyping Assay (Assay ID; C_{—}32174448_{—}10, manufactured by Applied Biosystems Inc.) specific to each polymorphism of rs11105364, and 0.45 μL of distilled water to 2.0 μL of the DNA solution obtained in Example 1, and was then provided for the extension reaction by a PCR method. The extension reaction was conducted through an initial incubation at 52° C. for 2 minutes and a subsequent incubation at 95° C. for 10 minutes, followed by 60 cycles consisting of heating at 95° C. for 15 seconds and at 60° C. for 1 minute. Following the extension reaction, genotyping of genetic polymorphisms was performed by measuring the fluorescence intensity using the 7900 HT Fast Real-Time PCR System (manufactured by Applied Biosystems Inc.).

### Example 21

### Correlation Between rs11105364 Polymorphism and High Blood Pressure

Correlation between the polymorphism identified in Example 20 and the high blood pressure among the individuals was analyzed by correlation analysis (association method). Table 19 shows the clinical backgrounds of 8,924 individuals subjected to the correlation analysis. These individuals were recruited in Yokohama (1,860 subjects), Shiga (3,953 subjects) and Ehime (3,539 subjects).

TABLE 19

| | |
|---|---|
| Age (years old) | 57 ± 14 |
| Sex (male/female) | 4828/4524 |
| Body mass index (kg/m²) | 23 ± 3 |
| Systolic blood pressure (mmHg) | 132 ± 20 |
| Diastolic blood pressure (mmHg) | 79 ± 12 |
| Antihypertensive medication (yes/no) | 1756/7594 |
| High blood pressure (yes/no) | 4014/5338 |
| Total cholesterol (mg/dL) | 202 ± 35 |
| HDL cholesterol (mg/dL) | 60 ± 15 |
| Neutral fat (mg/dL) | 117 ± 83 |
| Blood sugar (mg/dL) | 101 ± 27 |
| Amount of alcohol consumption (total) | 0.7 ± 1.0 |
| Smoking habit (yes/no) | 3287/6065 |
| Past history of cardiovascular diseases (yes/no) | 610/8742 |

The individuals shown in Table 19 were classified into a high blood pressure group (namely, a hypertension group having a systolic blood pressure of at least 140 mmHg and/or a diastolic blood pressure of at least 90 mmHg, and/or taking an antihypertensive agent) and a normal blood pressure group (namely, normotensive group other than those classified as the high blood pressure group), and allele frequencies of the polymorphisms identified in Example 20 were analyzed. A χ²-test was employed as a statistical analytical method. The results of the analysis are shown in Table 20.

TABLE 20

| | | | | Results of statistical analysis | | | |
|---|---|---|---|---|---|---|---|
| | Frequency of genetic | | | (upper box: odds ratio, lower box: p-value) | | | |
| | polymorphism | | | Allele | | | |
| | rs11105364 polymorphism | | | frequency | Frequency of genetic polymorphism | | |
| | TT | TG | GG | T/G | TT/TG + GG | TT + TG/GG | TT/TG/GG |
| High blood | 1706 | 1815 | 493 | 1.113 | 1.128 | 1.191 | — |
| pressure group | (42.5) | (45.2) | (12.3) | 0.001 | 0.005 | 0.005 | 0.002 |
| Normal blood | 2113 | 2462 | 763 | | | | |
| pressure group | (39.6) | (46.1) | (14.3) | | | | |

From the results described in Table 20, it became apparent that among the high blood pressure group and the normal blood pressure group, frequencies of each genotype with respect to the rs11105364 polymorphisms identified in Example 20 showed statistically significant difference. This result indicates that the relative risk for developing hypertension can be assessed by examining the rs11105364 polymorphism.

### Example 22

### Correlation Between rs11105364 Polymorphism and High Blood Pressure Analyzed by Logistic Regression Analysis

Correlation between the polymorphism identified in Example 20 and the high blood pressure among the general population was analyzed by a regression analysis that included other relevant environmental factors.

The individuals shown in Table 19 were classified into a high blood pressure group (namely, a hypertension group having a systolic blood pressure of at least 140 mmHg and/or a diastolic blood pressure of at least 90 mmHg, and/or taking an antihypertensive agent) and a normal blood pressure group (namely, normotensive group other than those classified as the high blood pressure group). A logistic regression analysis was carried out by using the above classification (namely, the high blood pressure group and the normal blood pressure group) as a dependent variable, while using sex, age, body mass index, past history of cardiovascular diseases, smoking habit, amount of alcohol consumption, HDL cholesterol, neutral fat, blood sugar and cohort, in addition to the polymorphism identified in Example 20, as independent variables. The results of the analysis are shown in Table 21.

TABLE 21

| | | Significance | Odds | Confidence |
|---|---|---|---|---|
| | | probability | ratio | interval |
| Age (years old) | | <0.001 | 1.081 | 1.076-1.086 |
| Sex (female) | | 0.070 | 1.134 | 0.990-1.298 |
| Body mass index (kg/m²) | | <0.001 | 1.201 | 1.181-1.222 |
| Past history of cardiovascular | | <0.001 | 1.530 | 1.251-1.871 |
| diseases | | | | |
| Smoking habit (yes/no) | | 0.094 | 0.897 | 0.789-1.019 |
| Amount of alcohol consumption | | <0.001 | 1.216 | 1.145-1.292 |
| (total) | | | | |
| HDL cholesterol (mg/dL) | | <0.001 | 1.013 | 1.009-1.017 |
| Neutral fat (mg/dL) | | <0.001 | 1.003 | 1.002-1.004 |
| Blood sugar (mg/dL) | | <0.001 | 1.005 | 1.003-1.007 |
| rs11105364 polymorphism | GG | Control | | |
| | TG | 0.022 | 1.189 | 1.026-1.379 |
| | TT | <0.001 | 1.341 | 1.154-1.557 |

From the results shown in Table 21, it became apparent that even after adjusted to other environmental factors such as sex, age, body mass index, past history of cardiovascular diseases, smoking habit, amount of alcohol consumption, HDL cholesterol, neutral fat, blood sugar and cohort variables, the polymorphism identified in Example 3 remained an independent risk factor for high blood pressure. In addition, the relative risk thereof (represented by odds ratio) was 1.189 fold for the TG genotype and 1.341 fold for the TT genotype, when compared to the GG genotype. These results indicate that the relative risk for developing hypertension can be assessed, even after adjusted to the effects of other environmental factors, by examining the rs11105364 polymorphism.

### Example 23

### Rough Calculation of Mean Blood Pressure for Each rs11105364 Polymorphism

Mean values of systolic blood pressure and diastolic blood pressure were roughly determined for each polymorphism identified in Example 20, and the differences therebetween were statistically analyzed by one-way analysis of variance. The results of the analysis are shown in Table 22.

TABLE 22

| | rs11105364 polymorphism | | | |
|---|---|---|---|---|
| | TT | TG | GG | |
| | (3819) | (4277) | (1256) | p-value |
| Systolic blood pressure | 133 ± 20 | 132 ± 20 | 130 ± 20 | <0.001 |
| (mmHg) | | | | |
| Diastolic blood pressure | 80 ± 12 | 79 ± 12 | 78 ± 11 | <0.001 |
| (mmHg) | | | | |

From the results shown in Table 22, it became apparent that mean values of systolic blood pressure and diastolic blood pressure determined roughly for each rs11105364 polymorphism differed statistically significantly. This result indicates that the degree of blood pressure elevation for each genetic polymorphism can be estimated by examining the rs11105364 polymorphism.

### Example 24

### Correlation Between rs11105364 Polymorphism and Blood Pressure Analyzed by Multiple Regression Analysis

Correlation between the polymorphism identified in Example 20 and the blood pressure among the general population was analyzed by a regression analysis that included other relevant environmental factors.

A multiple regression analysis was carried out by using systolic blood pressure or diastolic blood pressure as a dependent variable, while using sex, age, body mass index, past history of cardiovascular diseases, smoking habit, amount of alcohol consumption, use of antihypertensive medication, HDL cholesterol, neutral fat, blood sugar, and cohort variables, in addition to the polymorphism identified in Example 20, as independent variables. The results of the analysis are shown in Table 23.

TABLE 23

| | | Systolic blood pressure | | | Diastolic blood pressure | | |
|---|---|---|---|---|---|---|---|
| | | Unnormalized | Normalized | | Unnormalized | Normalized | |
| | | coefficient | coefficient | p-value | coefficient | coefficient | p-value |
| Age (years old) | | 0.513 | 0.342 | <0.001 | 0.174 | 0.200 | <0.001 |
| Sex (female) | | −2.938 | −0.072 | <0.001 | −3.889 | −0.165 | <0.001 |
| Body mass index (kg/m²) | | 1.266 | 0.194 | <0.001 | 0.917 | 0.242 | <0.001 |
| Past history of cardiovascular diseases | | −3.449 | −0.042 | <0.001 | −2.296 | −0.048 | <0.001 |
| (yes/no) | | | | | | | |
| Smoking habit (yes/no) | | −1.065 | −0.025 | 0.020 | −1.114 | −0.045 | <0.001 |
| Amount of alcohol consumption (total) | | 1.444 | 0.069 | <0.001 | 1.122 | 0.093 | <0.001 |
| Antihypertensive medication (yes/no) | | 10.631 | 0.205 | <0.001 | 4.044 | 0.134 | <0.001 |
| HDL cholesterol (mg/dL) | | 0.083 | 0.063 | <0.001 | 0.070 | 0.092 | <0.001 |
| Neutral fat (mg/dL) | | 0.020 | 0.080 | <0.001 | 0.016 | 0.110 | <0.001 |
| Blood sugar (mg/dL) | | 0.037 | 0.049 | <0.001 | 0.001 | 0.002 | 0.855 |
| rs11105364 polymorphism | GG | Control | | | Control | | |
| | TG | 1.645 | 0.040 | 0.002 | 0.875 | 0.037 | 0.008 |
| | TT | 2.733 | 0.066 | <0.001 | 1.417 | 0.059 | <0.001 |

From the results shown in Table 23, it became apparent that even after adjusted to other environmental factors such as sex, age, body mass index, past history of cardiovascular diseases, smoking habit, amount of alcohol consumption, use of antihypertensive medication, HDL cholesterol, neutral fat, blood sugar and cohort, the polymorphism identified in Example 20 remained an independent risk factor for systolic blood pressure or diastolic blood pressure.

This result indicates that the degree of blood pressure elevation for each genetic polymorphism can be estimated, even after adjusted to the relevant environmental factors shown in Table 23, by examining the rs11105364 polymorphism.

### Example 25

### Calculation of Adjusted Mean Blood Pressure for Each rs11105364 Polymorphism

From the regression analysis described in Example 24 which analyzed correlation between the polymorphism identified in Example 20 and blood pressure, mean values of systolic blood pressure and diastolic blood pressure for each rs11105364 polymorphism were calculated after adjusted to sex, age, body mass index, past history of cardiovascular diseases, smoking habit, amount of alcohol consumption, use of antihypertensive medication, HDL cholesterol, neutral fat, blood sugar and cohort (i.e., adjusted mean blood pressure). The results of the analysis are shown in Table 24.

TABLE 24

| | rs11105364 polymorphism | | | |
|---|---|---|---|---|
| | (mean ± standard error) | | | |
| | TT | TG | GG | |
| | (3819) | (4277) | (1256) | p-value |
| Systolic blood pressure | 133 ± 0.3 | 132 ± 0.3 | 130 ± 0.5 | <0.001 |
| (mmHg) | | | | |
| Diastolic blood pressure | 80 ± 0.2 | 79 ± 0.2 | 78 ± 0.3 | <0.001 |
| (mmHg) | | | | |

From the results shown in Table 24, it became apparent that adjusted mean values of systolic blood pressure and diastolic blood pressure determined for each rs11105364 polymorphism differed statistically significantly.

This result indicates that the degree of blood pressure elevation for each genetic polymorphism can be estimated, even after adjusted to the relevant environmental factors shown in Table 23, by examining the rs11105364 polymorphism.

### Example 26

### Genotyping of SNP (rs1799998) in CYP11B2 Gene

The SNP (rs1799998) in the CYP11B2 gene was analyzed by the TaqMan probe method using the amplified genomic DNA of each subject as a template. More specifically, a reaction solution was prepared by adding 2.5 μL of TaqMan Universal Master Mix (manufactured by Applied Biosystems Inc.), 0.05 μL of the TaqMan Pre-Designed SNP Genotyping Assay (Assay ID; C_{—}8896484_{—}10, manufactured by Applied Biosystems Inc.) specific to each polymorphism of rs1799998, and 0.45 μL of distilled water to 2.0 μL of the DNA solution obtained in Example 1, and was then provided for the extension reaction by a PCR method. The extension reaction was conducted through an initial incubation at 52° C. for 2 minutes and a subsequent incubation at 95° C. for 10 minutes, followed by 60 cycles consisting of heating at 95° C. for 15 seconds and at 60° C. for 1 minute. Following the extension reaction, genotyping of genetic polymorphisms was performed by measuring the fluorescence intensity using the 7900 HT Fast Real-Time PCR System (manufactured by Applied Biosystems Inc.),

### Example 27

### Genotyping of SNP (rs699) in AGT Gene

The SNP (rs699) in the AGT gene was analyzed by the TaqMan probe method using the amplified genomic DNA of each subject as a template. More specifically, a reaction solution was prepared by adding 2.5 μL of TaqMan Universal Master Mix (manufactured by Applied Biosystems Inc.), 0.05 μL of the TaqMan Pre-Designed SNP Genotyping Assay (Assay ID; C_{—}1985481_{—}20, manufactured by Applied Biosystems Inc.) specific to each polymorphism of rs699, and 0.45 μL of distilled water to 2.0 μL of the DNA solution obtained in Example 1, and was then provided for the extension reaction by a PCR method. The extension reaction was conducted through an initial incubation at 52° C. for 2 minutes and a subsequent incubation at 95° C. for 10 minutes, followed by 60 cycles consisting of heating at 95° C. for 15 seconds and at 60° C. for 1 minute. Following the extension reaction, genotyping of genetic polymorphisms was performed by measuring the fluorescence intensity using the 7900 HT Fast Real-Time PCR System (manufactured by Applied Biosystems Inc.).

### Example 28

### Correlation Between rs11105378 Polymorphism, rs1799998 Polymorphism, rs699 Polymorphism and High Blood Pressure Analyzed by Logistic Regression Analysis 1

Correlation between the rs11105378 polymorphism identified in Example 2, the rs1799998 polymorphism identified in Example 26, the rs699 polymorphism identified in Example 27 and high blood pressure was analyzed by a regression analysis that included other relevant environmental factors.

The individuals shown in Table 1 were classified into a high blood pressure group and a normal blood pressure group in the same manner as that described in Example 3. A logistic regression analysis was carried out by using the above classification (namely, the high blood pressure group and the normal blood pressure group) as a dependent variable, while using sex, age, body mass index, past history of cardiovascular diseases, smoking habit, amount of alcohol consumption, HDL cholesterol, neutral fat, blood sugar and cohort variables, in addition to the respective genetic polymorphisms identified in Examples 2, 26 and 27 as independent variables. The results of the analysis are shown in Table 25.

TABLE 25

| | | Significance | Odds | Confidence |
|---|---|---|---|---|
| | | probability | ratio | interval |
| Age (years old) | | <0.001 | 1.081 | 1.076-1.087 |
| Sex (female) | | 0.307 | 0.936 | 0.825-1.063 |
| Body mass index (kg/m²) | | <0.001 | 1.202 | 1.181-1.224 |
| Past history of cardiovascular | | <0.001 | 1.499 | 1.218-1.845 |
| diseases | | | | |
| Smoking habit (yes/no) | | 0.627 | 0.968 | 0.850-1.103 |
| Amount of alcohol consumption | | <0.001 | 1.208 | 1.135-1.285 |
| (total) | | | | |
| HDL cholesterol (mg/dL) | | <0.001 | 1.014 | 1.010-1.018 |
| Neutral fat (mg/dL) | | <0.001 | 1.003 | 1.002-1.004 |
| Blood sugar (mg/dL) | | <0.001 | 1.005 | 1.003-1.007 |
| rs11105378 polymorphism | TT | Control | | |
| | TC | 0.006 | 1.245 | 1.066-1.454 |
| | CC | <0.001 | 1.398 | 1.196-1.635 |
| rs699 polymorphism | MM | Control | | |
| | MT | 0.240 | 1.182 | 0.894-1.561 |
| | TT | 0.022 | 1.372 | 1.047-1.796 |
| rs1799998 polymorphism | CC | Control | | |
| | CT | 0.207 | 1.12 | 0.939-1.336 |
| | TT | 0.008 | 1.268 | 1.065-1.510 |

From the results shown in Table 25, it became apparent that even after adjusted to other environmental factors such as sex, age, body mass index, past history of cardiovascular diseases, smoking habit, amount of alcohol consumption, HDL cholesterol, neutral fat, blood sugar and cohort variables, the rs1799998 polymorphism identified in Example 26 and the rs699 polymorphism identified in Example 27 remained independent risk factors for high blood pressure. In addition, the relative risk thereof (represented by odds ratio) was 1.268 fold for the TT genotype when compared to the CC genotype with respect to the rs1799998 polymorphism, and 1.372 fold for the TT genotype when compared to the MM genotype with respect to the rs699 polymorphism.

From the above results, it is evident that the relative risk for developing hypertension can be assessed, even after adjusted to the effects of other environmental factors, by examining the rs1799998 polymorphism or the rs699 polymorphism. Moreover, it is also apparent from the above results that with respect to the SNP (rs1799998), those with the CC genotype or CT genotype may be classified into a low risk group whereas those with the TT genotype may be classified into a high risk group, and that with respect to the SNP (rs699), those with the MM genotype or MT genotype may be classified into a low risk group whereas those with the TT genotype may be classified into a high risk group.

### Example 29

### Correlation Between rs11105378 Polymorphism, rs1799998 Polymorphism, rs699 Polymorphism and High Blood Pressure Analyzed by Logistic Regression Analysis 2

By assigning 1 to the TC genotype and 2 to the CC genotype with respect to the rs11105378 polymorphism, 1 to the TT genotype with respect to the rs1799998 polymorphism, and 1 to the TT genotype with respect to the rs699 polymorphism, the number of risk polymorphisms present in each individual was calculated, and correlation between the number of risk polymorphisms and high blood pressure was analyzed by a regression analysis that included other relevant environmental factors.

More specifically, a logistic regression analysis was carried out in the same manner as that described in Example 28 except that the aforementioned number of risk polymorphisms present was used as a dependent variable instead of the respective genetic polymorphisms identified in Examples 2, 26 and 27. The results of the analysis are shown in Table 26.

TABLE 26

| | | Significance | Odds | Confidence |
|---|---|---|---|---|
| | | probability | ratio | interval |
| Age (years old) | | <0.001 | 1.081 | 1.076-1.087 |
| Sex (female) | | 0.317 | 0.937 | 0.826-1.064 |
| Body mass index (kg/m²) | | <0.001 | 1.202 | 1.180-1.223 |
| Past history of cardiovascular | | <0.001 | 1.498 | 1.217-1.843 |
| diseases | | | | |
| Smoking habit (yes/no) | | 0.621 | 0.968 | 0.849-1.102 |
| Amount of alcohol consumption | | <0.001 | 1.209 | 1.136-1.286 |
| (total) | | | | |
| HDL cholesterol (mg/dL) | | <0.001 | 1.014 | 1.010-1.017 |
| Neutral fat (mg/dL) | | <0.001 | 1.003 | 1.002-1.004 |
| Blood sugar (mg/dL) | | <0.001 | 1.005 | 1.003-1.007 |
| Number of risk | Absent/1 | Control | | |
| polymorphisms present | 2 | 0.005 | 1.231 | 1.063-1.426 |
| | 3 | <0.001 | 1.427 | 1.235-1.650 |
| | 4 | <0.001 | 1.641 | 1.377-1.956 |

From the results shown in Table 26, it became apparent that even after adjusted to other environmental factors such as sex, age, body mass index, past history of cardiovascular diseases, smoking habit, amount of alcohol consumption, HDL cholesterol, neutral fat, blood sugar and cohort variables, a combination of the respective genetic polymorphisms identified in Examples 2, 26 and 27 remained an independent risk factor for high blood pressure and showed higher relative risk (represented by odds ratio), as compare to those cases where the relative risk was assessed by examining each genetic polymorphism separately.

From the above results, it is evident that the relative risk for developing hypertension after adjusted to the effects of other environmental factors may be assessed more accurately by examining the combination of the rs11105378 polymorphism, the rs1799998 polymorphism and the rs699 polymorphism, rather than by assessing these genetic polymorphisms individually.

### Example 30

### Genotyping of SNP Using Polynucleotide for Detecting SNP (rs11105378)

By using the genomic DNA which was extracted from leucocytes in the peripheral blood collected from the individuals and was amplified in Example 1 as a template, genotyping of SNPs was carried out using the primers having the base sequences shown in Table 27.

TABLE 27

| | | Seq_ |
|---|---|---|
| Primer | Sequence | ID |
| SNP (rs11105378)_1st_Fw | GGCAGCTACACAGGTGTTCA | 1 |
| SNP (rs11105378)_1st_Rv | CGGGAAAACAGCAGTCATTT | 2 |
| SNP (rs11105378)_SS | GCTAGTCTGTTTTTCATGGC | 3 |
| Primer_Fw (C) | | |
| SNP (rs11105378)_SS | GCTAGTCTGTTTTTCATGGT | 4 |
| Primer_Fw (T) | | |
| SNP (rs11105378)_AS | GCTAGTCTGTTTTTCATGACA | 5 |
| Primer_Fw (C) | | |
| SNP (rs11105378)_AS | GCTAGTCTGTTTTTCATGATA | 6 |
| Primer_Fw (T) | | |
| SNP (rs11105378)_Rv | CGGGAAAACAGCAGTCATTT | 7 |

First, by using the genomic DNA extracted from each individual as a template, the genomic DNA was amplified using a forward primer for the first stage amplification having a base sequence assigned with a sequence number (Seq_ID) 1 (i.e., SNP (rs11105378)_{—}1st_Fw Primer)) and a reverse primer for the first stage amplification having a base sequence assigned with a sequence number 2 (i.e., SNP (rs11105378)_{—}1st_Rv Primer)). Subsequently, by using the obtained genomic DNA which was already amplified as described above as a template, PCR was carried out using a forward primer which may specifically detect the C allele of the SNP (rs11105378) and having a base sequence assigned with a sequence number 5 (i.e., SNP (rs11105378)_AS Primer_Fw (C)) or a forward primer which may specifically detect the T allele of the SNP (rs11105378) and having a base sequence assigned with a sequence number 6 (i.e., SNP (rs11105378)_AS Primer_Fw (T)) and a reverse primer having a base sequence assigned with a sequence number 7 (i.e., SNP (rs11105378)_Rv), thereby examining the presence and absence of PCR products by single molecule fluorescence analysis. It should be noted that the primers SNP (rs11105378)_AS Primer_Fw (C) and SNP (rs11105378)_AS Primer_Fw (T) were polynucleotides having a base sequence in which the SNP (rs11105378) was arranged at the second position from the 3′ end of the primer and a mismatch was introduced at the third position from the 3′ end of the primer.

More specifically, 20 μL of primary PCR solution was prepared by adding 2.0 μL of SNP (rs11105378)_{—}1st_Fw Primer (5 μM), 2.0 μL of SNP (rs11105378)_{—}1st_Rv Primer (5 μM), 1.0 μL of extracted genomic DNA (5 ng/μL) and 5 μL of sterile water to 10 μL of 2× AmpliTaq Gold Master Mix (manufactured by Applied Biosystems Inc.). Thereafter, genomic DNA was amplified by incubating the primary PCR solution at 95° C. for 10 minutes, followed by 40 thermal cycles consisting of 95° C. for 30 seconds, 57.5° C. for 30 seconds and 72° C. for 1 minute, and finally treating the resultant at 72° C. for 10 minutes.

Then, 20 μL of secondary PCR solution was prepared by adding 2 μL of 10× Stoffel Buffer (manufactured by Applied Biosystems Inc.), 1.6 μL of dNTP (10 mM), 2.0 μL of magnesium chloride solution (25 mM), 1.0 μL of the already amplified genomic DNA, 2.0 μL of TAMRA-labeled SNP (rs11105378)_AS Primer_Fw (C) (200 nM), 2.0 μL of Cy5-labeled SNP (rs11105378)_AS Primer_Fw (T) (200 nM), 2.0 μL of SNP (rs11105378)_Rv (200 nM), 0.1 μL of Stoffel fragment (10 units/μL, manufactured by Applied Biosystems Inc.) and an adequate amount of sterile water. Thereafter, the secondary PCR was carried out by incubating the secondary PCR solution at 95° C. for 2 minutes, followed by 40 thermal cycles consisting of 95° C. for 30 seconds, 63.2° C. for 30 seconds and 72° C. for 30 seconds, and finally treating the resultant at 72° C. for 10 minutes. It should be noted that the Gradient Thermal Cycler PTC-200 manufactured by MJ Research (now owned by Bio-Rad Laboratories, Inc.) was used as a PCR device.

The reaction solution obtained by the secondary PCR was poured into a glass plate used exclusively for a single molecule fluorescence analyzer MF20 (manufactured by Olympus Corporation), and was subjected to a simultaneous measurement of dual fluorescence at 543 nm and 633 nm as the measurement wavelengths. As a result, the presence and absence of PCR products was determined and genotyping of SNPs was carried out. The obtained results on genotyping of SNPs were the same as those obtained in Example 3.

In addition, genotyping of SNPs was carried out in the same manner as described above except that a forward primer having a base sequence assigned with a sequence number 3 in which the SNP (rs11105378) was arranged at the 3′ end of the primer (i.e., SNP (rs11105378)_SS Primer_Fw (C)) was used instead of the SNP (rs11105378)_AS Primer_Fw (C), and that a forward primer having a base sequence assigned with a sequence number 4 in which the SNP (rs11105378) was arranged at the 3′ end of the primer (i.e., SNP (rs11105378)_SS Primer_Fw (T)) was used instead of the SNP (rs11105378)_AS Primer_Fw (T). Again, the obtained results were the same as those obtained in Example 3.

From these results, it is apparent that genotyping of SNPs (in this case, the SNP (rs11105378)) may be carried out with high accuracy by using a polynucleotide for assessing the risk of developing hypertension according to the second aspect of the present invention.

### Example 31

### Genotyping of SNP Using Polynucleotide for Detecting SNP (rs2681472)

By using the genomic DNA which was extracted from leucocytes in the peripheral blood collected from the individuals and was amplified in Example 8 as a template, genotyping of SNPs was carried out using the primers having the base sequences shown in Table 28.

TABLE 28

| | | Seq_ |
|---|---|---|
| Primer | Sequence | ID |
| SNP (rs2681472)_1st_Fw | TCTGAGGATGTGGCATTTGA | 8 |
| SNP (rs2681472)_1st_Rv | TAGCCACACTGGCCTCTTTT | 9 |
| SNP (rs2681472)_SS | AGTGGGTCTGCCATGTAAAT | 10 |
| Primer_Fw (A) | | |
| SNP (rs2681472)_SS | AGTGGGTGTGCCATGTAAAC | 11 |
| Primer_Fw (G) | | |
| SNP (rs2681472)_AS | AGTGGGTCTGCCATGTAAGTA | 12 |
| Primer_Fw (A) | | |
| SNP (rs2681472)_AS | AGTGGGTCTGCCATGTAAGCA | 13 |
| Primer_Fw (G) | | |
| SNP (rs2681472)_Rv | TAGCCACACTGGCCTCTTTT | 14 |

First, by using the genomic DNA extracted from each individual as a template, the genomic DNA was amplified in the same manner as that described in Example 30 using a forward primer for the first stage amplification having a base sequence assigned with a sequence number 8 (i.e., SNP (rs2681472)_{—}1st_Fw Primer)) and a reverse primer for the first stage amplification having a base sequence assigned with a sequence number 9 (i.e., SNP (rs2681472)_{—}1st_Rv Primer)). Subsequently, by using the obtained genomic DNA which was already amplified as described above as a template, PCR was carried out in the same manner as that described in Example 30 using a forward primer which may specifically detect the A allele of the SNP (rs2681472) and having a base sequence assigned with a sequence number 12 (i.e., SNP (rs2681472)_AS Primer_Fw (A)) or forward primer which may specifically detect the G allele of the SNP (rs2681472) and having a base sequence assigned with a sequence number 13 (i.e., SNP (rs2681472)_AS Primer_Fw (G)) and a reverse primer having a base sequence assigned with a sequence number 14 (i.e., SNP (rs2681472)_Rv), thereby examining the presence and absence of PCR products by single molecule fluorescence analysis. The obtained results on genotyping of SNPs were the same as those obtained in Example 8. It should be noted that the primers SNP (rs2681472)_AS Primer_Fw (A) and SNP (rs2681472)_AS Primer_Fw (G) were polynucleotides having a base sequence in which the SNP (rs2681472) was arranged at the second position from the 3′ end of the primer and a mismatch was introduced at the third position from the 3′ end of the primer.

In addition, genotyping of SNPs was carried out in the same manner as described above except that a forward primer having a base sequence assigned with a sequence number 10 in which the SNP (rs2681472) was arranged at the 3′ end of the primer (i.e., SNP (rs2681472)_SS Primer_Fw (A)) was used instead of the SNP (rs2681472)_AS Primer_Fw (A), and that a forward primer having a base sequence assigned with a sequence number 11 in which the SNP (rs2681472) was arranged at the 3′ end of the primer (i.e., SNP (rs2681472)_SS Primer_Fw (G)) was used instead of the SNP (rs2681472)_AS Primer_Fw (G). Again, the obtained results were the same as those obtained in Example 8.

From these results, it is apparent that genotyping of SNPs (in this case, the SNP (rs2681472)) may be carried out with high accuracy by using a polynucleotide for assessing the risk of developing hypertension according to the third aspect of the present invention.

### Example 32

### Genotyping of SNP Using Polynucleotide for Detecting SNP (rs1401982)

By using the genomic DNA which was extracted from leucocytes in the peripheral blood collected from the individuals and was amplified in Example 14 as a template, genotyping of SNPs was carried out using the primers having the base sequences shown in Table 29.

TABLE 29

| | | Seq_ |
|---|---|---|
| Primer | Sequence | ID |
| SNP (rs1401982)_1st_Fw | TGTGGCTAGGGGAGCAGATA | 15 |
| SNP (rs1401982)_1st_Rv | AATGCTCCACCAACAAGGTT | 16 |
| SNP (rs1401982)_SS | CCTATGTTCTTGGAGTTATC | 17 |
| Primer_Fw (G) | | |
| SNP (rs1401982)_SS | CCTATGTTCTTGGAGTTATT | 18 |
| Primer_Fw (A) | | |
| SNP (rs1401982)_AS | CCTATGTTCTTGGAGTTACCC | 19 |
| Primer_Fw (G) | | |
| SNP (rs1401982)_AS | CCTATGTTCTTGGAGTTACTC | 20 |
| Primer_Fw (A) | | |
| SNP (rs1401982)_Rv | AATGCTCCACCAACAAGGTT | 21 |

First, by using the genomic DNA extracted from each individual as a template, the genomic DNA was amplified in the same manner as that described in Example 30 using a forward primer for the first stage amplification having a base sequence assigned with a sequence number 15 (i.e., SNP (rs1401982)_{—}1st_Fw Primer)) and a reverse primer for the first stage amplification having a base sequence assigned with a sequence number 16 (i.e., SNP (rs1401982)_{—}1st_Rv Primer)). Subsequently, by using the obtained genomic DNA which was already amplified as described above as a template, PCR was carried out in the same manner as that described in Example 30 using a forward primer which may specifically detect the G allele of the SNP (rs1401982) and having a base sequence assigned with a sequence number 19 (i.e., SNP (rs1401982)_AS Primer_Fw (G)) or forward primer which may specifically detect the A allele of the SNP (rs1401982) and having a base sequence assigned with a sequence number 20 (i.e., SNP (rs1401982)_AS Primer_Fw (A)) and a reverse primer having a base sequence assigned with a sequence number 21 (i.e., SNP (rs1401982)_Rv), thereby examining the presence and absence of PCR products by single molecule fluorescence analysis. The obtained results on genotyping of SNPs were the same as those obtained in Example 14. It should be noted that the primers SNP (rs1401982)_AS Primer_Fw (G) and SNP (rs1401982)_AS Primer_Fw (A) were polynucleotides having a base sequence in which the SNP (rs1401982) was arranged at the second position from the 3′ end of the primer and a mismatch was introduced at the third position from the 3′ end of the primer.

In addition, genotyping of SNPs was carried out in the same manner as described above except that a forward primer having a base sequence assigned with a sequence number 17 in which the SNP (rs1401982) was arranged at the 3′ end of the primer (i.e., SNP (rs1401982)_SS Primer_Fw (G)) was used instead of the SNP (rs1401982)_AS Primer_Fw (G), and that a forward primer having a base sequence assigned with a sequence number 18 in which the SNP (rs1401982) was arranged at the 3′ end of the primer (i.e., SNP (rs1401982)_SS Primer_Fw (A)) was used instead of the SNP (rs1401982)_AS Primer_Fw (A). Again, the obtained results were the same as those obtained in Example 14.

From these results, it is apparent that genotyping of SNPs (in this case, the SNP (rs1401982)) may be carried out with high accuracy by using a polynucleotide for assessing the risk of developing hypertension according to the fourth aspect of the present invention.

### Example 33

### Genotyping of SNP Using Polynucleotide for Detecting SNP (rs1799998)

By using the genomic DNA which was extracted from leucocytes in the peripheral blood collected from the individuals and was amplified in Example 2 as a template, genotyping of SNPs was carried out using the primers having the base sequences shown in Table 30.

TABLE 30

| | | Seq_ |
|---|---|---|
| Primer | Sequence | ID |
| SNP (rs1799998)_1st_Fw | TGGAGGGTGTACCTGTGTCA | 22 |
| SNP (rs1799998)_1st_Rv | TCCAGGGCTGAGAGGAGTAA | 23 |
| SNP (rs1799998)_SS | TATTAAAAGAATCCAAGGCT | 24 |
| Primer_Fw (T) | | |
| SNP (rs1799998)_SS | TATTAAAAGAATCCAAGGCC | 25 |
| Primer_Fw (C) | | |
| SNP (rs1799998)_AS | TATTAAAAGAATCCAAGGTTC | 26 |
| Primer_Fw (T) | | |
| SNP (rs1799998)_AS | TATTAAAAGAATCCAAGGTCC | 27 |
| Primer_Fw (C) | | |
| SNP (rs1799998)_Rv | TCCAGGGCTGAGAGGAGTAA | 28 |

First, by using the genomic DNA extracted from each individual as a template, the genomic DNA was amplified in the same manner as that described in Example 30 using a forward primer for the first stage amplification having a base sequence assigned with a sequence number 22 (i.e., SNP (rs1799998)_{—}1st_Fw Primer)) and a reverse primer for the first stage amplification having a base sequence assigned with a sequence number 23 (i.e., SNP (rs1799998)_{—}1st_Rv Primer)). Subsequently, by using the obtained genomic DNA which was already amplified as described above as a template, PCR was carried out in the same manner as that described in Example 30 using a forward primer which may specifically detect the T allele of the SNP (rs1799998) and having a base sequence assigned with a sequence number 26 (i.e., SNP (rs1799998)_AS Primer_Fw (T)) or forward primer which may specifically detect the C allele of the SNP (rs1799998) and having a base sequence assigned with a sequence number 27 (i.e., SNP (rs1799998)_AS Primer_Fw (C)) and a reverse primer having a base sequence assigned with a sequence number 28 (i.e., SNP (rs1799998)_Rv), thereby examining the presence and absence of PCR products by single molecule fluorescence analysis. The obtained results on genotyping of SNPs were the same as those obtained in Example 26. It should be noted that the primers SNP (rs1799998)_AS Primer_Fw (T) and SNP (rs1799998)_AS Primer_Fw (C) were polynucleotides having a base sequence in which the SNP (rs1799998) was arranged at the second position from the 3′ end of the primer and a mismatch was introduced at the third position from the 3′ end of the primer.

In addition, genotyping of SNPs was carried out in the same manner as described above except that a forward primer having a base sequence assigned with a sequence number 24 in which the SNP (rs1799998) was arranged at the 3′ end of the primer (i.e., SNP (rs1799998)_SS Primer_Fw (T)) was used instead of the SNP (rs1799998)_AS Primer_Fw (T), and that a forward primer having a base sequence assigned with a sequence number 25 in which the SNP (rs1799998) was arranged at the 3′ end of the primer (i.e., SNP (rs1799998)_SS Primer_Fw (C)) was used instead of the SNP (rs1799998)_AS Primer_Fw (C). Again, the obtained results were the same as those obtained in Example 26.

From these results, it is apparent that genotyping of SNPs (in this case, the SNP (rs1799998)) may be carried out with high accuracy by using a polynucleotide for assessing the risk of developing hypertension according to the sixth aspect of the present invention.

### Example 34

### Genotyping of SNP Using Polynucleotide for Detecting SNP (rs699)

By using the genomic DNA which was extracted from leucocytes in the peripheral blood collected from the individuals and was amplified in Example 2 as a template, genotyping of SNPs was carried out using the primers having the base sequences shown in Table 31.

TABLE 31

| | | Seq_ |
|---|---|---|
| Primer | Sequence | ID |
| SNP (rs699)_1st_Fw | GAACTGGATGTTGCTGCTGA | 29 |
| SNP (rs699)_1st_Rv | AGAGCCAGCAGAGAGGTTTG | 30 |
| SNP (rs699)_SS Primer_ | AAGACTGGCTGCTCCCTGAT | 31 |
| Fw (T) | | |
| SNP (rs699)_SS Primer_ | AAGACTGGCTGCTCCCTGAC | 32 |
| Fw (M) | | |
| SNP (rs699)_AS Primer_ | AAGACTGGCTGCTCCCTGGTG | 33 |
| Fw (T) | | |
| SNP (rs699)_AS Primer_ | AAGACTGGCTGCTCCCTGGCG | 34 |
| Fw (M) | | |
| SNP (rs699)_Rv | AGAGCCAGCAGAGAGGTTTG | 35 |

First, by using the genomic DNA extracted from each individual as a template, the genomic DNA was amplified in the same manner as that described in Example 30 using a forward primer for the first stage amplification having a base sequence assigned with a sequence number 29 (i.e., SNP (rs699)_{—}1st_Fw Primer)) and a reverse primer for the first stage amplification having a base sequence assigned with a sequence number 30 (i.e., SNP (rs699)_{—}1st_Rv Primer)). Subsequently, by using the obtained genomic DNA which was already amplified as described above as a template, PCR was carried out in the same manner as that described in Example 30 using a forward primer which may specifically detect the T allele of the SNP (rs699) and having a base sequence assigned with a sequence number 33 (i.e., SNP (rs699)_AS Primer_Fw (T)) or forward primer which may specifically detect the M allele of the SNP (rs699) and having a base sequence assigned with a sequence number 34 (i.e., SNP (rs699)_AS Primer Fw (M)) and a reverse primer having a base sequence assigned with a sequence number 35 (i.e., SNP (rs699)_Rv), thereby examining the presence and absence of PCR products by single molecule fluorescence analysis. The obtained results on genotyping of SNPs were the same as those obtained in Example 27. It should be noted that the primers SNP (rs699)_AS Primer_Fw (T) and SNP (rs699)_AS Primer_Fw (M) were polynucleotides having a base sequence in which the SNP (rs699) was arranged at the second position from the 3′ end of the primer and a mismatch was introduced at the third position from the 3′ end of the primer.

In addition, genotyping of SNPs was carried out in the same manner as described above except that a forward primer having a base sequence assigned with a sequence number 31 in which the SNP (rs699) was arranged at the 3′ end of the primer (i.e., SNP (rs699)_SS Primer_Fw (T)) was used instead of the SNP (rs699)_AS Primer_Fw (T), and that a forward primer having a base sequence assigned with a sequence number 32 in which the SNP (rs699) was arranged at the 3′ end of the primer (i.e., SNP (rs699)_SS Primer_Fw (M)) was used instead of the SNP (rs699)_AS Primer_Fw (M). Again, the obtained results were the same as those obtained in Example 27.

From these results, it is apparent that genotyping of SNPs (in this case, the SNP (rs699)) may be carried out with high accuracy by using a polynucleotide for assessing the risk of developing hypertension according to the sixth aspect of the present invention.

### Example 35

### Other Methods for Assessing SNP

It is also possible to assess the risk of developing hypertension by collecting, apart from the aforementioned SNP (rs11105378), the SNP (rs2681472), the SNP (rs1401982) and the SNP (rs11105364) of the ATP2B1 gene, the SNP (rs1799998) of the CYP11B2 gene and the SNP (rs699) of the AGT gene which are highly correlated with high blood pressure, a plurality of SNPs that have low correlation with high blood pressure and calculating the risk of developing hypertension due to the presence of the plurality of SNPs in the form of scores, thereby analyzing the correlation between high blood pressure and the obtained scores.

The technique involves the following procedures.

1. A SNP is classified into a risk genotype, a hetero genotype, and a non-risk genotype, based on the risk of developing hypertension, and scores of 3, 2 and 1 are assigned to the above genotypes, respectively.

2. A plurality of SNPs are classified in the same manner as described above, based on the risk of developing hypertension, and the obtained scores are summed together so as to calculate a risk value.

3. The risk value as defined above is determined for each individual in the sample population, and a histogram is drawn by calculating (frequency)×(risk value).

4. The obtained histogram is divided into a high risk group, an intermediate risk group, and a low risk group.

5. The SNPs of a subject are examined and the subject is classified into the high risk group, the intermediate group, or the low risk group, by comparing the scores obtained for the subject with the histogram.

In the present example, as the SNPs having low correlation with high blood pressure, among those 38 SNPs disclosed in the aforementioned Patent Document 5, a SNP (rs2070759) of the ATP2B1 and 11 other SNPs from different genes were used to carry out the assessment. The 12 SNPs used for the assessment are listed in Table 32.

TABLE 32

| Gene | SNP | AA | Aa | aa | Risk genotype |
|---|---|---|---|---|---|
| ATP2B1 | rs2070759 | GG | GT | TT | TT |
| DLGAP2 | rs2301963 | CC | CA | AA | CC |
| RAC2 | rs929023 | TT | TC | CC | TT |
| SLC22A7 | rs2270860 | GG | GA | AA | AA |
| HLADMB | rs2071556 | CC | CA | AA | CC |
| KCNN1 | rs2278993 | TT | TC | CC | TT |
| PRKWNK1 | rs2255390 | GG | GA | AA | GG |
| PTHR1 | rs1869872 | TT | TC | CC | CC |
| GUCA1C | rs2715709 | GG | GA | AA | AA |
| ACCN1 | rs28933 | GG | GA | AA | AA |
| FGF2 | rs3747676 | GG | GA | AA | GG |
| ATP2A3 | rs887387 | TT | TC | CC | TT |

Based on the genotypes obtained from 8,467 subjects, scores of 3, 2 and 1 were assigned to the risk genotype, the hetero genotype, and the non-risk genotype, respectively, and the total score of each subject was calculated and shown in the histogram in Table 33.

TABLE 33

| | Number of | Percentage of number of |
|---|---|---|
| Score | subjects | subjects |
| 15 | 3 | 0.00035432 |
| 16 | 11 | 0.00129916 |
| 17 | 34 | 0.00401559 |
| 18 | 111 | 0.01310972 |
| 19 | 277 | 0.03271525 |
| 20 | 546 | 0.06448565 |
| 21 | 884 | 0.10440534 |
| 22 | 1198 | 0.14149049 |
| 23 | 1369 | 0.16168655 |
| 24 | 1294 | 0.15282863 |
| 25 | 1148 | 0.13558521 |
| 26 | 780 | 0.09212236 |
| 27 | 452 | 0.05338373 |
| 28 | 225 | 0.02657376 |
| 29 | 101 | 0.01192866 |
| 30 | 27 | 0.00318885 |
| 31 | 7 | 0.00082674 |

In Table 33, scores of 26 or more are defined as the high risk group, scores of 20 or less are defined as the low risk group, and scores between 21 and 25 are defined as the intermediate risk group.

Among the above-mentioned 8,467 subjects, those in a high blood pressure group (namely, a group having (systolic blood pressure)/(diastolic blood pressure) of at least 160 mmHg/90 mmHg, and/or taking an antihypertensive agent; 1,655 subjects) and those in a normal blood pressure group (namely, a group having (systolic blood pressure)/(diastolic blood pressure) of less than 120 mmHg/90 mmHg, and/or taking no antihypertensive agent; 1,786 subjects) were compared with the frequencies of high risk group/intermediate risk group/low risk group. Comparison results are shown in Table 34.

TABLE 34

| | | Normal blood | High blood | |
|---|---|---|---|---|
| | | pressure group | pressure group | Total |
| Low risk group | Frequency | 204 | 136 | 340 |
| | Total % | 7.04 | 4.69 | 11.73 |
| | Column % | 13.63 | 9.71 | |
| | Row % | 60.00 | 40.00 | |
| Intermediate | Frequency | 1043 | 967 | 2010 |
| risk group | Total % | 35.99 | 33.37 | 69.36 |
| | Column % | 69.67 | 69.02 | |
| | Row % | 51.89 | 48.11 | |
| High risk group | Frequency | 250 | 298 | 548 |
| | Total % | 8.63 | 10.28 | 18.91 |
| | Column % | 16.70 | 21.27 | |
| | Row % | 45.62 | 54.38 | |
| Total | Frequency | 1497 | 1401 | 2898 |
| | Total % | 51.66 | 48.34 | 100 |

In Table 34, among the subjects (i.e., 1,786 subjects in the normal blood pressure group and 1,655 subjects in the high blood pressure group), genotyping results of all 12 SNPs were successfully achieved for 1,497 subjects in the normal blood pressure group and 1,401 subjects in the high blood pressure group. Here, the p-value was p=0.0002. The value is obtained when the test is performed based on a null hypothesis assuming that the frequencies of high risk group/intermediate risk group/low risk group are the same for those in the high blood pressure group and those in the normal blood pressure group. The result means that the frequencies of high risk group/intermediate risk group/low risk group are different for those in the high blood pressure group and those in the normal blood pressure group at a probability of 99.9998%.

Correlation between the aforementioned 12 SNPs and high blood pressure was analyzed by a regression analysis that included other relevant environmental factors. The subjects were classified into a high risk group, an intermediate risk group, and a low risk group. A logistic regression analysis was carried out by using the degree of risks classified here as a dependent variable, while using sex, age and body mass index as independent variables. The results of the analysis are shown in Table 35.

TABLE 35

| | p-value | Odds ratio | 95% confidence interval |
|---|---|---|---|
| Age | 0.884 | 1.001 | 0.991-1.011 |
| Sex (female) | <0.001 | 0.412 | 0.349-0.486 |
| BMI | <0.001 | 1.360 | 1.319-1.403 |
| Low risk group | Control | | |
| Medium risk group | 0.066 | 1.275 | 0.984-1.655 |
| High risk group | 0.001 | 1.675 | 1.234-2.274 |

From the results shown in Table 35, it became apparent that even after adjusted to the environmental factors such as sex, age and body mass index, the risk of developing hypertension can be assessed by combining and integrating the results of the SNP (rs2070759) in the ATP2B1 gene and other 11 SNPs in different genes. In addition, the relative risk thereof (represented by odds ratio) was 1.275 fold for the intermediate risk group and 1.675 fold for the high risk group, when compared to the low risk group. Because the genes used here have low correlation with high blood pressure apart from the ATP2B1 gene, it is important to combine and integrate the results of a plurality of polymorphisms. Genotypes of the subjects in the high blood pressure group and in the normal blood pressure group are shown in Table 36.

TABLE 36

| | | High | | | Normal | | | |
|---|---|---|---|---|---|---|---|---|
| | | blood pressure group | | | blood pressure group | | | |
| Gene | SNP | AA | Aa | aa | AA | Aa | aa | p-value |
| ATP2B1 | rs2070759 | 325 | 769 | 513 | 439 | 838 | 459 | 0.0001 |
| DLGAP2 | rs2301963 | 443 | 770 | 421 | 463 | 834 | 458 | 0.8902 |
| RAC2 | rs929023 | 337 | 797 | 489 | 336 | 858 | 558 | 0.3927 |
| SLC22A7 | rs2270860 | 662 | 754 | 203 | 769 | 772 | 205 | 0.1796 |
| HLADMB | rs2071556 | 444 | 796 | 387 | 453 | 825 | 470 | 0.1156 |
| KCNN1 | rs2278993 | 161 | 713 | 767 | 159 | 742 | 870 | 0.3462 |
| PRKWNK1 | rs2255390 | 427 | 781 | 417 | 411 | 859 | 461 | 0.2375 |
| PTHR1 | rs1869872 | 334 | 790 | 512 | 349 | 837 | 566 | 0.8102 |
| GUCA1C | rs2715709 | 673 | 747 | 189 | 769 | 759 | 200 | 0.2784 |
| ACCN1 | rs28933 | 386 | 835 | 394 | 437 | 882 | 416 | 0.6883 |
| FGF2 | rs3747676 | 359 | 812 | 429 | 397 | 818 | 500 | 0.1852 |
| ATP2A3 | rs887387 | 810 | 664 | 160 | 838 | 754 | 175 | 0.4358 |

In Table 36, the values for AA, Aa, and aa are the same as those in Table 32. In addition, as in the case of Table 34, the p-value was obtained when the frequencies of AA, Aa, and aa are compared between those in the high blood pressure group and those in the normal blood pressure group.

### Example 36

### ATP2B1 Gene Knockout Mice in the Vascular Smooth Muscle Cells

In order to clarify the importance of ATP2B1 gene in blood pressure regulation, we attempt to knockout the function of the ATP2B1 gene with organ specificity. Here, we deleted the ATP2B1 gene in the vascular smooth muscle cells of mice, which were referred as “VSMC ATP2B1 KO” mice. To generate the conditional ATP2B1 KO mice, we utilized the Cre/loxP and FLP-FRT recombination system.

### <Animal Care>

Animals were housed under a 12-hour day/night cycle at a temperature of 25° C. Tap water was provided ad libitum. Experiments were conducted under the guidelines for animal experiments set by the Animal Experiment Committee of Yokohama City University School of Medicine.

### <Cre-Mice>

SM22-Cre mice [Tg(Tagln-cre)1Her/J, stock #004746] were obtained from The Jackson Laboratory (Bar Harbor, Me., USA). The SM22-Cre transgenic mice express Cre recombinase under control of the mouse transgelin (smooth muscle protein 22-alpha) promoter. Thus SM22-Cre mice are knocked out the gene that is sandwiched with loxP sites in vascular smooth muscle cell (VSMC) specifically. Mice engineered in this study were backcrossed onto the (C57BL/6) genetic background for at least nine generations.

### <Generation of ATP2B1 Floxed Mice>

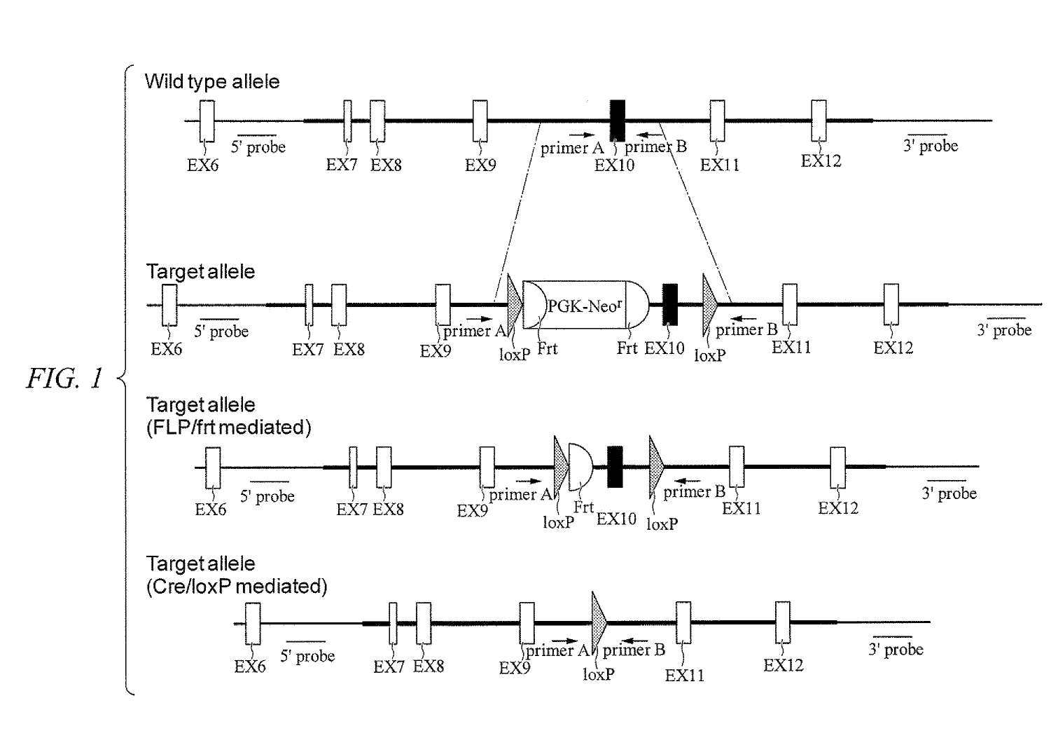

ATP2B1 is encoded by 21 exons on chromosome 10. As previously reported (Okunade et al., J Biol Chem. 2004 Aug. 6, vol. 279(32), p 33742-50), mice lacking exon 10 of ATP2B1 could not survive for birth, suggesting that the exon 10 might be a flagship region of ATP2B1 gene. Thus, we designed a new vector to knockout the exon 10 of ATP2B1 gene with the Cre/loxP and FLP-FRT recombination system. An FRT-PGK-neo-FRTloxP cassette was sandwiched upstream of exon 10 and a loxP site was sandwiched downstream of exon 10, resulting in a foxed fragment containing the exon 10 of ATP2B1 gene (FIG. 1). The targeting vector contains 5572 by homologous region of ATP2B1 upstream of the FRT-PGK-neo-FRTloxP cassette and 3891 by homologous region downstream the loxP site. The targeting vector was linearized, and electroporated into the C57BL/6 (RENKA) ES cell line (Transgenic Laboratory, Kumamoto, Japan). Neo-resistant ES cell clones (n=143) were screened for 5′probe, 3′probe and neo probe with Kpn I digestion and for 3′probe and neo probe with Ovu II digestion by using Southern blot analysis. Six positive clones were detected and re-analyzed using primer A having a base sequence assigned with a sequence number 36 and primer B having a base sequence assigned with a sequence number 37 for confirming the presence of exon 10 and loxP sequences. These primers' base sequences were shown in Table 37. The Flp gene expression vector was electroporated into the cloned ES cells to eliminate the PGK-neo region. Three ATP2B1_{loxP/+} ES cell clones were expanded and aggregated with embryos (ICR strain) to generate chimeras. The chimeras were mated to detect germline transmission. The resulting ATP2B1_{loxP/+} mice were mated to generate foxed ATP2B1 allele (ATP2B1_{loxP/loxP}) mice.

TABLE 37

| | | Seq_ |
|---|---|---|
| Primer | Sequence | ID |
| Primer A | ATCCTGTCCTACCTGGTAAC | 36 |
| Primer B | GTAGAACCCTGACCTAACAG | 37 |

### <Creation of Smooth Muscle Cell-Targeted ATP2B1 KO Mouse Lines>

To target the inactivation of the ATP2B1 gene to VSMC, ATP2B1_{loxP/loxP} mice were intercrossed with SM22-Cre transgenic mice expressing Cre recombinase under control of the mouse transgelin (smooth muscle protein 22-alpha) promoter. The resulting ATP2B1_{loxP}/SM22-Cre animals were further mated with ATP2B1_{loxP/loxP} mice to generate ATP2B1_{loxP/loxP}/SM22-Cre (VSMC ATP2B1 KO) mice.

<Quantification of ATP2B1 mRNA Expression by Real-Time Reverse Transcription (RT)-PCR.>

Real-time quantitative RT-PCR was performed to determine levels of ATP2B1 mRNA expression of aortas from foxed ATP2B1 and VSMC ATP2B1 KO mice. Total RNA was isolated by the acid guanidinium thiocyanate-phenol-chloroform extraction method. RT reactions were performed using SuperScript III reverse transcriptase (Invitrogen, Burlington, ON, Canada). Quantitative PCR analysis was conducted by incubating RT product with TaqMan universal PCR master mix and specific primer-probe set of ATP2B1 (product No. Mn01245796_ml, Applied Biosystems, Foster City, Calif.); the PCR reaction was run on an ABI Prism 7500 detection system using standard conditions. RNA quantity was expressed relative to an 18S endogenous control. Relative expression levels were expressed by the comparative threshold cycle (Ct) method.

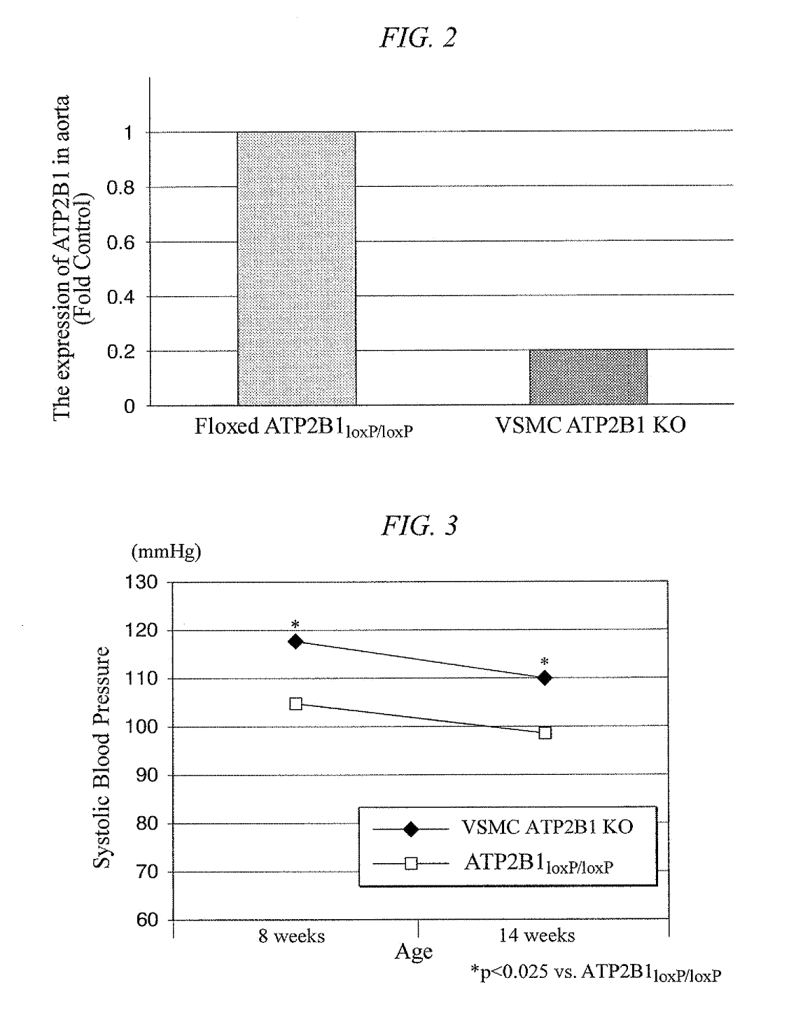

Southern blot analysis of tail DNA obtained from the VSMC ATP2B1 KO mice demonstrated the deletion event, which occurs in VSMC within the vascular bed of the tail. Quantitative RT-PCR analysis demonstrated that the expression of ATP2B1 mRNA in isolated aortas of VSMC ATP2B1 KO mice was reduced by 80%, compared to homozygous foxed ATP2B1_{loxP,loxP} mice without Cre recombinase (FIG. 2).

To ascertain whether deletion of ATP2B1 gene in vascular smooth muscle cells affects their blood pressure, conscious homozygous foxed ATP2B1_{loxP/loxP} and VSMC ATP2B1 KO mice were subjected to blood pressure measurements by tail cuff methods. All experiments were carried out in a blinded fashion on male mice eating a standard rodent chow (0.5% NaCl). Remarkably, under resting conditions, VSMC ATP2B1 KO mice displayed higher systolic blood pressure values than homozygous foxed ATP2B1_{loxP/loxP} mice in 8 weeks and 14 weeks of age (FIG. 3). On the other hand, heart rates did not differ significantly between the two groups.

### INDUSTRIAL APPLICABILITY

By using the genetic marker for hypertension according to the present invention, the risk of developing hypertension may be assessed more accurately, and thus the present invention can be used in various fields including the gene analysis of specimens in medical institutions or the like.

What is claimed is:

1. A genetic marker for hypertension comprising:
a sequence homologous to or complementary to a partial or complete sequence of an ATP2B1 gene which contains a single nucleotide polymorphism (SNP) of the ATP2B1 gene,
wherein the SNP is at least one SNP selected from the group consisting of a SNP (rs11105378), a SNP (rs2681472), a SNP (rs1401982), and a SNP (rs11105364).

2. The genetic marker for hypertension according to claim 1,
wherein the SNP is at least one SNP selected from the group consisting of a SNP (rs11105378) and a SNP (rs2681472).

3. The genetic marker for hypertension according to claim 1,
wherein the SNP is at least one SNP selected from the group consisting of a SNP (rs11105378) and a SNP (rs1401982).

4. The genetic marker for hypertension according to claim 1,
wherein the SNP is at least one SNP selected from the group consisting of a SNP (rs2681472) and a SNP (rs1401982).

5. The genetic marker for hypertension according to claim 1, wherein the SNP is a SNP (rs11105378).

6. The genetic marker for hypertension according to claim 1, wherein the SNP is a SNP (rs2681472).

7. The genetic marker for hypertension according to claim 1, wherein the SNP is a SNP (rs1401982).

8. The genetic marker for hypertension according to claim 1, wherein the SNP is a SNP (rs11105364).

9. A polynucleotide for assessing the risk of developing hypertension comprising:
any one of the following base sequences (a) to (f),
wherein the polynucleotide can be used as a primer or probe for detecting a SNP (rs11105378):
(a) a base sequence represented by sequence number 5 or a base sequence which is a partial sequence of the base sequence represented by sequence number 5 containing the SNP (rs11105378);
(b) a base sequence complementary to the base sequence (a);
(c) a base sequence composed of the base sequence (a) or (b) in which 1 or more bases other than the SNP (rs11105378) are deleted, substituted, or added, wherein the polynucleotide including the base sequence can be hybridized with the polynucleotide including the base sequence (a) or (b) under stringent conditions;
(d) a base sequence represented by sequence number 6 or a base sequence which is a partial sequence of the base sequence represented by sequence number 6 containing the SNP (rs11105378);
(e) a base sequence complementary to the base sequence (d);
(f) a base sequence composed of the base sequence (d) or (e) in which 1 or more bases other than the SNP (rs11105378) are deleted, substituted, or added, wherein the polynucleotide including the base sequence can be hybridized with the polynucleotide including the base sequence (d) or (e) under stringent conditions.

10. A polynucleotide for assessing the risk of developing hypertension comprising:
any one of the following base sequences (a) to (f),
wherein the polynucleotide can be used as a primer or probe for detecting a SNP (rs2681472):
(a) a base sequence represented by sequence number 12 or a base sequence which is a partial sequence of the base sequence represented by sequence number 12 containing the SNP (rs2681472);
(b) a base sequence complementary to the base sequence (a);
(c) a base sequence composed of the base sequence (a) or (b) in which 1 or more bases other than the SNP (rs2681472) are deleted, substituted, or added, wherein the polynucleotide including the base sequence can be hybridized with the polynucleotide including the base sequence (a) or (b) under stringent conditions;
(d) a base sequence represented by sequence number 13 or a base sequence which is a partial sequence of the base sequence represented by sequence number 13 containing the SNP (rs2681472);
(e) a base sequence complementary to the base sequence (d);
(f) a base sequence composed of the base sequence (d) or (e) in which 1 or more bases other than the SNP (rs2681472) are deleted, substituted, or added, wherein the polynucleotide including the base sequence can be hybridized with the polynucleotide including the base sequence (d) or (e) under stringent conditions.

11. A polynucleotide for assessing the risk of developing hypertension comprising:
any one of the following base sequences (a) to (f),
wherein the polynucleotide can be used as a primer or probe for detecting a SNP (rs1401982):
(a) a base sequence represented by sequence number 19 or a base sequence which is a partial sequence of the base sequence represented by sequence number 19 containing the SNP (rs1401982);
(b) a base sequence complementary to the base sequence (a);
(c) a base sequence composed of the base sequence (a) or (b) in which 1 or more bases other than the SNP (rs1401982) are deleted, substituted, or added, wherein the polynucleotide including the base sequence can be hybridized with the polynucleotide including the base sequence (a) or (b) under stringent conditions;
(d) a base sequence represented by sequence number 20 or a base sequence which is a partial sequence of the base sequence represented by sequence number 20 containing the SNP (rs1401982);
(e) a base sequence complementary to the base sequence (d);
(f) a base sequence composed of the base sequence (d) or (e) in which 1 or more bases other than the SNP (rs1401982) are deleted, substituted, or added, wherein the polynucleotide including the base sequence can be hybridized with the polynucleotide including the base sequence (d) or (e) under stringent conditions.

12. A genetic marker for hypertension comprising a sequence homologous to or complementary to a partial or complete sequence of a CYP11B2 gene containing a SNP (rs1799998) which is a SNP of the CYP11B2 gene.

13. A polynucleotide for assessing the risk of developing hypertension comprising:
any one of the following base sequences (a) to (f),
wherein the polynucleotide can be used as a primer or probe for detecting a SNP (rs1799998):
(a) a base sequence represented by sequence number 26 or a base sequence which is a partial sequence of the base sequence represented by sequence number 26 containing the SNP (rs1799998);
(b) a base sequence complementary to the base sequence (a);
(c) a base sequence composed of the base sequence (a) or (b) in which 1 or more bases other than the SNP (rs1799998) are deleted, substituted, or added, wherein the polynucleotide including the base sequence can be hybridized with the polynucleotide including the base sequence (a) or (b) under stringent conditions;
(d) a base sequence represented by sequence number 27 or a base sequence which is a partial sequence of the base sequence represented by sequence number 27 containing the SNP (rs1799998);
(e) a base sequence complementary to the base sequence (d);
(f) a base sequence composed of the base sequence (d) or (e) in which 1 or more bases other than the SNP (rs1799998) are deleted, substituted, or added, wherein the polynucleotide including the base sequence can be hybridized with the polynucleotide including the base sequence (d) or (e) under stringent conditions.

14. A method for assessing the risk of developing hypertension by using a genetic marker, the method comprising:
(a) a step of genotyping at least one SNP selected from the group consisting of a SNP (rs11105378), a SNP (rs2681472), a SNP (rs1401982), a SNP (rs11105364) and a SNP (rs1799998) which are present in nucleic acid molecules collected from a human individual; and
(b) a step of assessing the risk for the human individual to develop hypertension based on the genotyping result obtained in the step (a).

15. The method for assessing the risk of developing hypertension according to claim 14,
wherein the step (a) is a step further genotyping a SNP (rs699) which is a SNP of an AGT gene.

16. The method for assessing the risk of developing hypertension according to claim 14,
wherein in the step (b), with respect to the SNP (rs11105378), the risk of developing hypertension is assessed to be highest for a CC genotype, followed by a TC genotype and a TT genotype in this order.

17. The method for assessing the risk of developing hypertension according to claim 14,
wherein in the step (b), with respect to the SNP (rs2681472), the risk of developing hypertension is assessed to be highest for an AA genotype, followed by an AG genotype and a GG genotype in this order.

18. The method for assessing the risk of developing hypertension according to claim 14,
wherein in the step (b), with respect to the SNP (rs1401982), the risk of developing hypertension is assessed to be highest for a GG genotype, followed by an AG genotype and an AA genotype in this order.

19. The method for assessing the risk of developing hypertension according to claim 14,
wherein in the step (b), with respect to the SNP (rs11105364), the risk of developing hypertension is assessed to be highest for a TT genotype, followed by a TG genotype and a GG genotype in this order.

20. The method for assessing the risk of developing hypertension according to claim 14,
wherein in the step (b), with respect to the SNP (rs11105378), those with a TT genotype are assessed as a low risk group whereas those with a TC genotype or a CC genotype are assessed as a high risk group.

21. The method for assessing the risk of developing hypertension according to claim 14,
wherein in the step (b), with respect to the SNP (rs2681472), those with a GG genotype or an AG genotype are assessed as a low risk group whereas those with an AA genotype are assessed as a high risk group.

22. The method for assessing the risk of developing hypertension according to claim 14,
wherein in the step (b), with respect to the SNP (rs1401982), those with an AA genotype are assessed as a low risk group whereas those with an AG genotype or a GG genotype are assessed as a high risk group.

23. The method for assessing the risk of developing hypertension according to claim 14,
wherein in the step (b), with respect to the SNP (rs11105364), those with a GG genotype are assessed as a low risk group whereas those with a TT genotype or a TG genotype are assessed as a high risk group.

24. The method for assessing the risk of developing hypertension according to claim 14,
wherein in the step (b), with respect to the SNP (rs1799998), those with a CC genotype or a CT genotype are assessed as a low risk group whereas those with a TT genotype are assessed as a high risk group.

25. The method for assessing the risk of developing hypertension according to claim 15,
wherein in the step (b), with respect to the SNP (rs699), those with an MM genotype or an MT genotype are assessed as a low risk group (with the proviso that M stands for methionine (Met) and T stands for threonine (Thr)), whereas those with a TT genotype are assessed as a high risk group.

26. The method for assessing the risk of developing hypertension according to claim 14,
wherein in the step (b), those with a TT genotype with respect to the SNP (rs11105378) and a CC genotype or a CT genotype with respect to the SNP (rs1799998) are assessed as a low risk group, whereas those with a TC genotype or a CC genotype with respect to the SNP (rs11105378) and a TT genotype with respect to the SNP (rs1799998) are assessed as a high risk group.

27. The method for assessing the risk of developing hypertension according to claim 14,
wherein in the step (b), those with a GG genotype or an AG genotype with respect to the SNP (rs2681472) and a CC genotype or a CT genotype with respect to the SNP (rs1799998) are assessed as a low risk group, whereas those with an AA genotype with respect to the SNP (rs2681472) and a TT genotype with respect to the SNP (rs1799998) are assessed as a high risk group.

28. The method for assessing the risk of developing hypertension according to claim 14,
wherein in the step (b), those with an AA genotype with respect to the SNP (rs1401982) and a CC genotype or a CT genotype with respect to the SNP (rs1799998) are assessed as a low risk group, whereas those with an AG genotype or a GG genotype with respect to the SNP (rs1401982) and a TT genotype with respect to the SNP (rs1799998) are assessed as a high risk group.

29. The method for assessing the risk of developing hypertension according to claim 15,
wherein in the step (b), those with a TT genotype with respect to the SNP (rs11105378) and an MM genotype or an MT genotype with respect to the SNP (rs699) (with the proviso that M stands for methionine (Met) and T stands for threonine (Thr)) are assessed as a low risk group, whereas those with a TC genotype or a CC genotype with respect to the SNP (rs11105378) and a TT genotype with respect to the SNP (rs699) are assessed as a high risk group.

30. The method for assessing the risk of developing hypertension according to claim 15,
wherein in the step (b), those with a GG genotype or an AG genotype with respect to the SNP (rs2681472) and an MM genotype or an MT genotype with respect to the SNP (rs699) (with the proviso that M stands for methionine (Met) and T stands for threonine (Thr)) are assessed as a low risk group, whereas those with an AA genotype with respect to the SNP (rs2681472) and a TT genotype with respect to the SNP (rs699) are assessed as a high risk group.

31. The method for assessing the risk of developing hypertension according to claim 15,
wherein in the step (b), those with an AA genotype with respect to the SNP (rs1401982) and an MM genotype or an MT genotype with respect to the SNP (rs699) (with the proviso that M stands for methionine (Met) and T stands for threonine (Thr)) are assessed as a low risk group, whereas those with an AG genotype or a GG genotype with respect to the SNP (rs1401982) and a TT genotype with respect to the SNP (rs699) are assessed as a high risk group.

32. The method for assessing the risk of developing hypertension according to claim 15,
wherein in the step (b), those with a CC genotype or a CT genotype with respect to the SNP (rs1799998) and an MM genotype or an MT genotype with respect to the SNP (rs699) (with the proviso that M stands for methionine (Met) and T stands for threonine (Thr)) are assessed as a low risk group, whereas those with a TT genotype with respect to the SNP (rs1799998) and a TT genotype with respect to the SNP (rs699) are assessed as a high risk group.

33. The method for assessing the risk of developing hypertension according to claim 15,
wherein in the step (b), those with a TT genotype with respect to the SNP (rs11105378), a CC genotype or a CT genotype with respect to the SNP (rs1799998) and an MM genotype or an MT genotype with respect to the SNP (rs699) (with the proviso that M stands for methionine (Met) and T stands for threonine (Thr)) are assessed as a low risk group, whereas those with a TC genotype or a CC genotype with respect to the SNP (rs11105378), a TT genotype with respect to the SNP (rs1799998) and a TT genotype with respect to the SNP (rs699) are assessed as a high risk group.

34. The method for assessing the risk of developing hypertension according to claim 15,
wherein in the step (b), those with a GG genotype or an AG genotype with respect to the SNP (rs2681472), a CC genotype or a CT genotype with respect to the SNP (rs1799998) and an MM genotype or an MT genotype with respect to the SNP (rs699) (with the proviso that M stands for methionine (Met) and T stands for threonine (Thr)) are assessed as a low risk group, whereas those with an AA genotype with respect to the SNP (rs2681472), a TT genotype with respect to the SNP (rs1799998) and a TT genotype with respect to the SNP (rs699) are assessed as a high risk group.

35. The method for assessing the risk of developing hypertension according to claim 15,
wherein in the step (b), those with an AA genotype with respect to the SNP (rs1401982), a CC genotype or a CT genotype with respect to the SNP (rs1799998) and an MM genotype or an MT genotype with respect to the SNP (rs699) (with the proviso that M stands for methionine (Met) and T stands for threonine (Thr)) are assessed as a low risk group, whereas those with an AG genotype or a GG genotype with respect to the SNP (rs1401982), a TT genotype with respect to the SNP (rs1799998) and a TT genotype with respect to the SNP (rs699) are assessed as a high risk group.

36. The method for assessing the risk of developing hypertension according to claim 15, further comprising:
a step of classifying a CC genotype with respect to the SNP (rs11105378), a TT genotype with respect to the SNP (rs1799998) and a TT genotype with respect to the SNP (rs699) (with the proviso that T stands for threonine (Thr)) as high risk polymorphisms,
wherein the risk of developing hypertension is assessed, in the step (b), to be highest when the number of the high risk polymorphisms present is 3, followed by the cases where the number of the high risk polymorphisms present is 2, 1 and 0 in this order.

37. The method for assessing the risk of developing hypertension according to claim 15, further comprising:
a step of classifying an AA genotype with respect to the SNP (rs2681472), a TT genotype with respect to the SNP (rs1799998) and a TT genotype with respect to the SNP (rs699) (with the proviso that T stands for threonine (Thr)) as high risk polymorphisms,
wherein the risk of developing hypertension is assessed, in the step (b), to be highest when the number of the high risk polymorphisms present is 3, followed by the cases where the number of the high risk polymorphisms present is 2, 1 and 0 in this order.

38. The method for assessing the risk of developing hypertension according to claim 15, further comprising:
a step of classifying a GG genotype with respect to the SNP (rs1401982), a TT genotype with respect to the SNP (rs1799998) and a TT genotype with respect to the SNP (rs699) (with the proviso that T stands for threonine (Thr)) as high risk polymorphisms,
wherein the risk of developing hypertension is assessed, in the step (b), to be highest when the number of the high risk polymorphisms present is 3, followed by the cases where the number of the high risk polymorphisms present is 2, 1 and 0 in this order.

39. The method for assessing the risk of developing hypertension according to claim 15, further comprising:
a step of classifying a TT genotype with respect to the SNP (rs11105378), a CC genotype with respect to the SNP (rs1799998) and an MM genotype with respect to the SNP (rs699) (with the proviso that M stands for methionine (Met)) as low risk polymorphisms,
wherein, the risk of developing hypertension is assessed, in the step (b), to be highest when the number of the low risk polymorphisms present is 0, followed by the cases where the number of the low risk polymorphisms present is 1, 2 and 3 in this order.

40. The method for assessing the risk of developing hypertension according to claim 15, further comprising:
a step of classifying a GG genotype with respect to the SNP (rs2681472), a CC genotype with respect to the SNP (rs1799998) and an MM genotype with respect to the SNP (rs699) (with the proviso that M stands for methionine (Met)) as low risk polymorphisms,
wherein the risk of developing hypertension is assessed, in the step (b), to be highest when the number of the low risk polymorphisms present is 0, followed by the cases where the number of the low risk polymorphisms present is 1, 2 and 3 in this order.

41. The method for assessing the risk of developing hypertension according to claim 15, further comprising:
a step of classifying an AA genotype with respect to the SNP (rs1401982), a CC genotype with respect to the SNP (rs1799998) and an MM genotype with respect to the SNP (rs699) (with the proviso that M stands for methionine (Met)) as low risk polymorphisms,
wherein the risk of developing hypertension is assessed, in the step (b), to be highest when the number of the low risk polymorphisms present is 0, followed by the cases where the number of the low risk polymorphisms present is 1, 2 and 3 in this order.

42. The method for assessing the risk of developing hypertension according to claim 15, further comprising:
a step of classifying those with a TT genotype with respect to the SNP (rs11105378), a CC genotype or a CT genotype with respect to the SNP (rs1799998) and an MM genotype or an MT genotype with respect to the SNP (rs699) (with the proviso that M stands for methionine (Met) and T stands for threonine (Thr)) as a first group;
a step of classifying those with a TC genotype or a CC genotype with respect to the SNP (rs11105378), a CC genotype or a CT genotype with respect to the SNP (rs1799998) and an MM genotype or an MT genotype with respect to the SNP (rs699), those with a TT genotype with respect to the SNP (rs11105378), a CC genotype or a CT genotype with respect to the SNP (rs1799998) and a TT genotype with respect to the SNP (rs699), or those with a TT genotype with respect to the SNP (rs11105378), a TT genotype with respect to the SNP (rs1799998) and an MM genotype or an MT genotype with respect to the SNP (rs699) as a second group;
a step of classifying those with a TC genotype or a CC genotype with respect to the SNP (rs11105378), a CC genotype or a CT genotype with respect to the SNP (rs1799998) and a TT genotype with respect to the SNP (rs699), those with a TC genotype or a CC genotype with respect to the SNP (rs11105378), a TT genotype with respect to the SNP (rs1799998) and an MM genotype or an MT genotype with respect to the SNP (rs699), or those with a TT genotype with respect to the SNP (rs11105378), a TT genotype with respect to the SNP (rs1799998) and a TT genotype with respect to the SNP (rs699) as a third group; and
a step of classifying those with the TC genotype or CC genotype with respect to the SNP (rs11105378), the TT genotype with respect to the SNP (rs1799998) and the TT genotype with respect to the SNP (rs699) as a fourth group,
wherein in the step (b), the risk of developing hypertension is assessed to be highest for the fourth group, followed by the third group, the second group and the first group in this order.

43. The method for assessing the risk of developing hypertension according to claim 15, further comprising:
a step of classifying those with a GG genotype or an AG genotype with respect to the SNP (rs2681472), a CC genotype or a CT genotype with respect to the SNP (rs1799998) and an MM genotype or an MT genotype with respect to the SNP (rs699) (with the proviso that M stands for methionine (Met) and T stands for threonine (Thr)) as a first group;
a step of classifying those with an AA genotype with respect to the SNP (rs2681472), a CC genotype or a CT genotype with respect to the SNP (rs1799998) and an MM genotype or an MT genotype with respect to the SNP (rs699), those with a GG genotype or an AG genotype with respect to the SNP (rs2681472), a CC genotype or a CT genotype with respect to the SNP (rs1799998) and a TT genotype with respect to the SNP (rs699), or those with a GG genotype or an AG genotype with respect to the SNP (rs2681472), a TT genotype with respect to the SNP (rs1799998) and an MM genotype or an MT genotype with respect to the SNP (rs699) as a second group;
a step of classifying those with an AA genotype with respect to the SNP (rs2681472), a CC genotype or a CT genotype with respect to the SNP (rs1799998) and a TT genotype with respect to the SNP (rs699), those with an AA genotype with respect to the SNP (rs2681472), a TT genotype with respect to the SNP (rs1799998) and an MM genotype or an MT genotype with respect to the SNP (rs699), or those with a GG genotype or an AG genotype with respect to the SNP (rs2681472), a TT genotype with respect to the SNP (rs1799998) and a TT genotype with respect to the SNP (rs699) as a third group; and
a step of classifying those with an AA genotype with respect to the SNP (rs2681472), a TT genotype with respect to the SNP (rs1799998) and a TT genotype with respect to the SNP (rs699) as a fourth group,
wherein in the step (b), the risk of developing hypertension is assessed to be highest for the fourth group, followed by the third group, the second group and the first group in this order.

44. The method for assessing the risk of developing hypertension according to claim 15, further comprising:
a step of classifying those with an AA genotype with respect to the SNP (rs1401982), a CC genotype or a CT genotype with respect to the SNP (rs1799998) and an MM genotype or an MT genotype with respect to the SNP (rs699) (with the proviso that M stands for methionine (Met) and T stands for threonine (Thr)) as a first group;
a step of classifying those with an AG genotype or a GG genotype with respect to the SNP (rs1401982), a CC genotype or a CT genotype with respect to the SNP (rs1799998) and an MM genotype or an MT genotype with respect to the SNP (rs699), those with an AA genotype with respect to the SNP (rs1401982), a CC genotype or a CT genotype with respect to the SNP (rs1799998) and a TT genotype with respect to the SNP (rs699), or those with an AA genotype with respect to the SNP (rs1401982), a TT genotype with respect to the SNP (rs1799998) and an MM genotype or an MT genotype with respect to the SNP (rs699) as a second group;
a step of classifying those with an AG genotype or a GG genotype with respect to the SNP (rs1401982), a CC genotype or a CT genotype with respect to the SNP (rs1799998) and a TT genotype with respect to the SNP (rs699), those with an AG genotype or a GG genotype with respect to the SNP (rs1401982), a TT genotype with respect to the SNP (rs1799998) and an MM genotype or an MT genotype with respect to the SNP (rs699), or those with an AA genotype with respect to the SNP (rs1401982), a TT genotype with respect to the SNP (rs1799998) and a TT genotype with respect to the SNP (rs699) as a third group; and
a step of classifying those with an AG genotype or a GG genotype with respect to the SNP (rs1401982), a TT genotype with respect to the SNP (rs1799998) and a TT genotype with respect to the SNP (rs699) as a fourth group,
wherein in the step (b), the risk of developing hypertension is assessed to be highest for the fourth group, followed by the third group, the second group and the first group in this order.

45. The method for assessing the risk of developing hypertension according to claim 14, further comprising:
making an assessment on the risk for developing hypertension by combining genotyping results obtained in the step (a) with at least one risk factor of the human individual selected from the group consisting of sex, age, body mass index (BMI), the presence of cerebrovascular disease, the presence of cardiac disease, smoking habit, amount of alcohol consumption, total cholesterol, high-density lipoprotein (HDL) cholesterol, neutral fat, and fasting blood sugar.

46. A microarray for assessing the risk of developing hypertension comprising:
a solid support; and
at least one polynucleotide selected from the group consisting of the polynucleotides of claims 9, 10, 11 and 13 for assessing the risk of developing hypertension, which is fixed onto the solid support.

47. The microarray for assessing the risk of developing hypertension according to claim 46, further comprising
a polynucleotide which includes any one of the following base sequences (a) to (f) and which can be used as a primer or probe for detecting a SNP (rs699), and which is also fixed onto the solid support:
(a) a base sequence represented by sequence number 33 or a base sequence which is a partial sequence of the base sequence represented by sequence number 33 containing the SNP (rs699);
(b) a base sequence complementary to the base sequence (a);
(c) a base sequence composed of the base sequence (a) or (b) in which 1 or more bases other than the SNP (rs699) are deleted, substituted, or added, wherein the polynucleotide including the base sequence can be hybridized with the polynucleotide including the base sequence (a) or (b) under stringent conditions;
(d) a base sequence represented by sequence number 34 or a base sequence which is a partial sequence of the base sequence represented by sequence number 34 containing the SNP (rs699);
(e) a base sequence complementary to the base sequence (d);
(f) a base sequence composed of the base sequence (d) or (e) in which 1 or more bases other than the SNP (rs699) are deleted, substituted, or added, wherein the polynucleotide including the base sequence can be hybridized with the polynucleotide including the base sequence (d) or (e) under stringent conditions.

48. A SNP genotyping kit for assessing the risk of developing hypertension comprising at least one selected from the group consisting of
the polynucleotide of claim 9 for assessing the risk of developing hypertension,
the polynucleotide of claim 10 for assessing the risk of developing hypertension,
the polynucleotide of claim 11 for assessing the risk of developing hypertension, and
the polynucleotide of claim 13 for assessing the risk of developing hypertension.

49. The SNP genotyping kit for assessing the risk of developing hypertension according to claim 48, further comprising
a polynucleotide which includes any one of the following base sequences (a) to (f) and which can be used as a primer or probe for detecting a SNP (rs699):
(a) a base sequence represented by sequence number 33 or a base sequence which is a partial sequence of the base sequence represented by sequence number 33 containing the SNP (rs699);
(b) a base sequence complementary to the base sequence (a);
(e) a base sequence composed of the base sequence (a) or (b) in which 1 or more bases other than the SNP (rs699) are deleted, substituted, or added, wherein the polynucleotide including the base sequence can be hybridized with the polynucleotide including the base sequence (a) or (b) under stringent conditions;
(d) a base sequence represented by sequence number 34 or a base sequence which is a partial sequence of the base sequence represented by sequence number 34 containing the SNP (rs699);
(e) a base sequence complementary to the base sequence (d);
(f) a base sequence composed of the base sequence (d) or (e) in which 1 or more bases other than the SNP (rs699) are deleted, substituted, or added, wherein the polynucleotide including the base sequence can be hybridized with the polynucleotide including the base sequence (d) or (e) under stringent conditions.

50. A loxP integrated vector comprising:
a base sequence in which the entire ATP2B1 gene sequence or a portion thereof is sandwiched between loxP sequences,
wherein the loxP integrated vector is used for constructing a small animal in which an ATP2B1 gene is locally deleted.

51. A loxP integrated small animal,
wherein the loxP integrated small animal is constructed using the loxP integrated vector of claim 50 and is used for constructing a small animal in which an ATP2B1 gene is locally deleted.

52. A small animal in which an ATP2B1 gene is locally deleted,
wherein the small animal is constructed by:
crossing a transgenic small animal selectively expressing Cre Recombinase having at least one promoter selected from the group consisting of a Tie-2 promoter, a Tie-1 promoter, an Flk-1 promoter, an SM22 promoter, an SM-MHC promoter, a Wt1 promoter, a P0 promoter, a Pax3 promoter, an αMHC promoter, an Nkx2.5 promoter, a Tbx1 promoter, a tetracycline-inducible promoter, and a CMV enhancer-chicken β-actin promoter as an expression promoter for the Cre Recombinase,
with the loxP integrated small animal of claim 51.

53. The small animal in which an ATP2B1 gene is locally deleted according to claim 52, wherein the small animal is exhibiting a symptom of hypertension.

54. A method for using a small animal in which an ATP2B1 gene is locally deleted, the method comprising
using the small animal of claim 53 in which an ATP2B1 gene is locally deleted as a test animal for screening calcium antagonists.

55. A method for using a small animal in which an ATP2B1 gene is locally deleted, the method comprising
using the small animal of claim 53 in which an ATP2B1 gene is locally deleted as a small animal model for disorders caused by a genetic polymorphism and impaired expression concerning the ATP2B1 gene.

56. The method for using a small animal in which an ATP2B1 gene is locally deleted according to claim 55,
wherein the genetic polymorphism is at least one SNP selected from the group consisting of a SNP (rs11105378), a SNP (rs2681472), a SNP (rs1401982), and a SNP (rs11105364).
